(19) United States
(12) Patent Application Publication
(10) Pub. No.: US 2022/0145292 A1
(43) Pub. Date: May 12, 2022

(54) LIGAND CLUSTERS AND METHODS OF THEIR USE AND PREPARATION

(71) Applicant: Deep Genomics Incorporated, Toronto (CA)

(72) Inventor: Jovanka BOGOJESKI, Toronto (CA)

(21) Appl. No.: 17/434,460

(22) PCT Filed: Feb. 28, 2020
(86) PCT No.: PCT/CA2020/050272

§ 371 Date: Aug. 27, 2021

Related U.S. Application Data
(60) Provisional application No. 62/812,201, filed on Feb. 28, 2019.

(51) Int. Cl. C12N 15/11; A61K 47/54

(52) U.S. Cl. CPC C12N 15/111; C07H 21/00; A61K 47/549

(57) ABSTRACT

Disclosed are compounds of formula (I): Yₚ—X-L²-Z, (I) or a salt thereof, where p is 1 to 5; X is a monosaccharide; each Y is independently -L¹-T, H, protecting group, optionally substituted hydrocarbon, or optionally substituted heteroorganic group, wherein each T is independently a ligand or a protected ligand, and each L¹ is independently a covalent linker; L2 is a conjugation linker; Z is a therapeutically active agent, protecting group, or a conjugation moiety. Also disclosed are methods of use of the compounds of the invention and methods of their preparation.

### FIELD OF THE INVENTION

The invention relates to ligand clusters and methods of their use and preparation.

### BACKGROUND

Therapeutic applications often suffer from off-target effects associated with the delivery of a therapeutically active agent to an off-target cell or tissue. Targeting moiety-based approaches have been in development to address the problem of off-target effects with varying degrees of success.

There is a need for new targeting moieties and, in particular, for oligonucleotides having a new targeting moiety.

### SUMMARY OF THE INVENTION

In general, the invention provides compounds that are useful for targeting cells, e.g., in a tissue, e.g., in a subject, and intermediates useful in the synthesis thereof. The compounds of the invention include a targeting moiety of the following structure:

Yₚ—X-L²-,

where
p is 1 to 5;
X is a monosaccharide;
each Y is independently -L¹-T, H, protecting group, optionally substituted hydrocarbon, or optionally substituted heteroorganic group, where each T is independently a ligand or a protected ligand, and each L¹ is independently a covalent linker; and
L² is a conjugation linker;
provided that at least one Y is -L¹-T.

In one aspect, the invention provides a compound of formula (I):

Yₚ—X-L²-Z, (I)

or a salt thereof,
where
p is 1 to 5;
X is a monosaccharide;
each Y is independently -L¹-T, H, protecting group, optionally substituted hydrocarbon, or optionally substituted heteroorganic group, where each T is independently a ligand or a protected ligand, and each L¹ is independently a covalent linker;
L² is a conjugation linker; and
Z is a therapeutically active agent, protecting group, or a conjugation moiety;
provided that at least one Y is -L¹-T.

In some embodiments, the monosaccharide is a pentose or hexose, where,
when the monosaccharide is a pentose, p is 1 to 3, and
when the monosaccharide is a hexose, p is 1 to 4.

In certain embodiments, the monosaccharide is N-acetylgalactosamine, galactosamine, galactose, mannose, allose, altrose, glucose, gulose, idose, talose, arabinose, lyxose, ribose, or xylose. In particular embodiments, the monosaccharide is N-acetylgalactosamine.

In further embodiments, the group -L²-Z is a group of the following structure:

-Q¹-Q²-Z,

where
Q¹ is [-Q³-Q⁴-Q⁵]ₛ-Q^{C}-B¹, where B¹ is a bond to Q²;
Q² is [-Q³-Q⁴-Q⁵]ₛ-B², where B² is a bond to Z;
each Q³ is independently absent, —CO—, —NH—, —O—, —S—, —SO₂—, —OC(O)—, —C(O)O—, —NHC(O)—, —C(O)NH—, —CH₂—, —CH₂NH—, —NHCH₂—, —CH₂O—, or —OCH₂—;
each Q⁴ is independently absent, optionally substituted C₁₋₁₂ alkylene, optionally substituted C₂₋₁₂ alkenylene, optionally substituted C₂₋₁₂ alkynylene, optionally substituted C₂₋₁₂ heteroalkylene, optionally substituted C₆₋₁₀ arylene, optionally substituted C₁₋₉ heteroarylene, or optionally substituted C₁₋₉ heterocyclylene;
each Q⁵ is independently absent, —CO—, —NH—, —O—, —S—, —SO₂—, —CH₂—, —C(O)O—, —OC(O)—, —C(O)NH—, —NHC(O)—, —NH—CH(R^{a})—C(O)—, —C(O)—CH(R^{a})—NH—, —OP(O)(OH)O—, or —OP(S)(OH)O—, where each R^{a} is independently H or optionally substituted C₁₋₁₂ alkyl;
Q^{C} is optionally substituted C₂₋₁₂ alkylene, optionally substituted C₂₋₁₂ heteroalkylene, optionally substituted C₁₋₁₂ thioheterocyclylene, optionally substituted C₁₋₁₂ heterocyclylene, cyclobut-3-ene-1,2-dione-3,4-diyl, pyrid-2-yl hydrazone, optionally substituted C₆₋₁₆ triazoloheterocyclylene, optionally substituted C₈₋₁₆ triazolocycloalkenylene, or a dihydropyridazine group; and
each s is independently 0 to 20.

In certain preferred embodiments, Q^{C} is optionally substituted C₂₋₁₂ heteroalkylene, optionally substituted C₁₋₁₂ thioheterocyclylene, optionally substituted C₁₋₁₂ heterocyclylene, cyclobut-3-ene-1,2-dione-3,4-diyl, pyrid-2-yl hydrazone, optionally substituted C₆₋₁₆ triazoloheterocyclylene, optionally substituted C₈₋₁₆ triazolocycloalkenylene, or a dihydropyridazine group.

In particular preferred embodiments, each Q⁵ is independently absent, —CO—, —NH—, —O—, —S—, —SO₂—, —CH₂—, —C(O)O—, —OC(O)—, —C(O)NH—, —NHC(O)—, —OP(O)(OH)O—, or —OP(S)(OH)O—.

In yet further embodiments, -L²-Z is a group of the following structure:

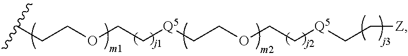

where
each of m1 and m2 is independently 0, 1, 2, 3, 4, 5, 6, 7, 8, 9, or 10; and
each of j1, j2, and j3 is independently 1, 2, 3, 4, or 5.

In still further embodiments, each Q⁵ is independently —NHC(O)— or —C(O)NH—.

In some embodiments, -L²-Z is a group of the following structure:

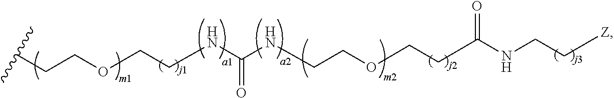

where a1 is 0 and a2 is 1, or a1 is 1 and a2 is 0.

In certain embodiments, -L²-Z is a group of the following structure:

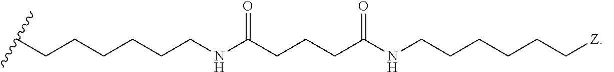

In some embodiments, -L²-Z is a group of the following structure:

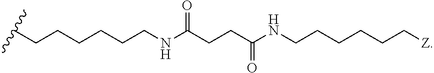

In particular embodiments, Z is a therapeutically active agent.

In further embodiments, the therapeutically active agent is a therapeutically active oligonucleotide.

In yet further embodiments, the therapeutically active oligonucleotide is an antisense oligonucleotide, splice-switching oligonucleotide, siRNA, miRNA, or CpG ODN.

In still further embodiments, -L²-Z is a group of the following structure:

[-Q³-Q⁴-Q⁵]ₛ-Z

where
s is 1 to 20;
each Q³ is independently absent, —CO—, —NH—, —O—, —S—, —SO₂—, —OC(O)—, —C(O)O—, —NHC(O)—, —C(O)NH—, —CH₂—, —CH₂NH—, —NHCH₂—, —CH₂O—, or —OCH₂—;
each Q⁴ is independently absent, optionally substituted C₁₋₁₂ alkylene, optionally substituted C₂₋₁₂ alkenylene, optionally substituted C₂₋₁₂ alkynylene, optionally substituted C₂₋₁₂ heteroalkylene, optionally substituted C₆₋₁₀ arylene, optionally substituted C₁₋₉ heteroarylene, or optionally substituted C₁₋₉ heterocyclylene;
each Q⁵ is independently absent, —CO—, —NH—, —O—, —S—, —SO₂—, —CH₂—, —C(O)O—, —OC(O)—, —C(O)NH—, —NHC(O)—, —NH—CH(R^{a})—C(O)—, —C(O)—CH(R^{a})—NH—, —OP(O)(OH)O—, or —OP(S)(OH)O—, where each R^{a} is independently H or optionally substituted C₁₋₁₂ alkyl; and
provided that at least one Q⁴ is present.

In particular preferred embodiments, each Q⁵ is independently absent, —CO—, —NH—, —O—, —S—, —SO₂—, —CH₂—, —C(O)O—, —OC(O)—, —C(O)NH—, —NHC(O)—, —OP(O)(OH)O—, or —OP(S)(OH)O—.

In some embodiments, -L²-Z is a group of the following structure:

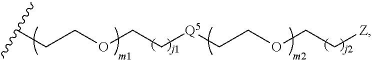

where
each of m1 and m2 is independently 0, 1, 2, 3, 4, 5, 6, 7, 8, 9, or 10; and
each of j1 and j2 is independently 1, 2, 3, 4, or 5.

In some embodiments, -L²-Z is a group of the following structure:

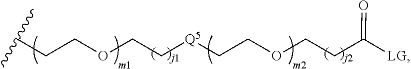

where
LG is a leaving group.

In certain embodiments, the leaving group is pentafluorophenoxy or tetrafluorophenoxy.

In particular embodiments, -L²-Z is a group of the following structure:

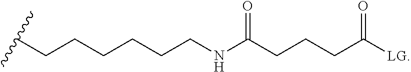

In some embodiments, -L²-Z is a group of the following structure:

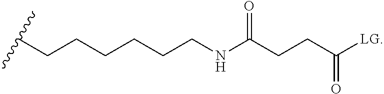

In further embodiments, each -L¹-T is independently a group of the following structure:

[-Q³-Q⁴-Q⁵]ₛ-Q6-T,

where
s is 0 to 20;
each Q³ and each Q⁶ are independently absent, —CO—, —NH—, —O—, —S—, —SO₂—, —OC(O)—, —C(O)O—, —NHC(O)—, —C(O)NH—, —CH₂—, —CH₂NH—, —NHCH₂—, —CH₂O—, or —OCH₂—;
each Q⁴ is independently absent, optionally substituted C₁₋₁₂ alkylene, optionally substituted C₂₋₁₂ alkenylene, optionally substituted C₂₋₁₂ alkynylene, optionally substituted C₂₋₁₂ heteroalkylene, optionally substituted C₆₋₁₀ arylene, optionally substituted C₁₋₉ heteroarylene, or optionally substituted C₁₋₉ heterocyclylene; and
each Q⁵ is independently absent, —CO—, —NH—, —O—, —S—, —SO₂—, —CH₂—, —C(O)O—, —OC(O)—, —C(O)NH—, —NHC(O)—, —NH—CH(R^{a})—C(O)—, —C(O)—CH(R^{a})—NH—, —OP(O)(OH)O—, or —OP(S)(OH)O—, where each R^{a} is independently H or optionally substituted C₁₋₁₂ alkyl;
provided that at least one of Q³, Q⁴, Q⁵, and Q⁶ is present.

In particular preferred embodiments, each Q⁵ is independently absent, —CO—, —NH—, —O—, —S—, —SO₂—, —CH₂—, —C(O)O—, —OC(O)—, —C(O)NH—, —NHC(O)—, —OP(O)(OH)O—, or —OP(S)(OH)O—.

In yet further embodiments, s is 1, 2, 3, 4, 5, 6, 7, 8, 9, or 10.

In still further embodiments, each -L¹-T is independently a group of the following structure:

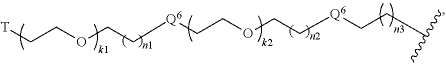

where
each of k1 and k2 is independently 0, 1, 2, 3, 4, 5, 6, 7, 8, 9, or 10; and
each of n1, n2, and n3 is independently 1, 2, 3, 4, or 5.

In some embodiments, each Q⁶ is independently —NHC(O)— or —C(O)NH—.

In certain embodiments, each -L¹-T is independently a group of the following structure:

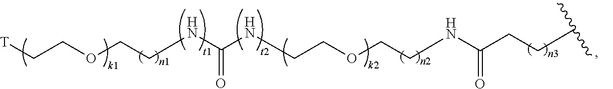

where t1 is 0 and t2 is 1, or t1 is 1 and t2 is 0.

In particular embodiments, each -L¹-T is a group of the following structure:

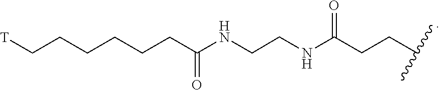

In further embodiments, each -L¹-T is a group of the following structure:

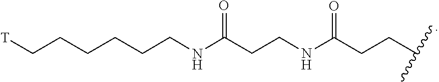

In yet further embodiments, each T is independently a ligand.

In still further embodiments, each T is N-acetylgalactosamine.

In some embodiments, each T is independently a protected ligand.

In certain embodiments, each T is N-acetylgalactosamine triacetate.

In some embodiments, the compound is of the following structure:

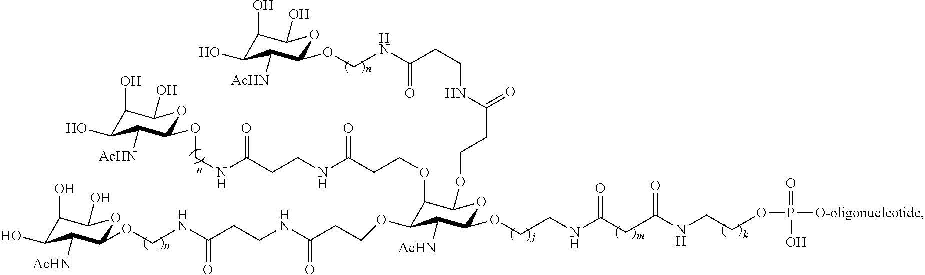

or a salt thereof, where each n is independently 1 to 20, j is 1 to 11, k is 1 to 11, and m is 1 to 10.

In some embodiments, j is 5.

In certain embodiments, k is 5.

In further embodiments, m is 2 or 3.

In yet further embodiments, the compound is of the following structure:

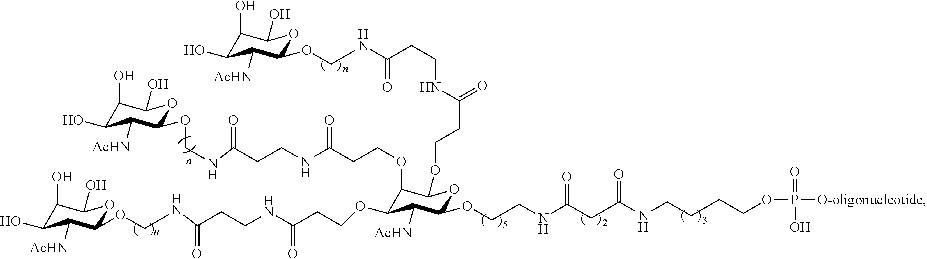

or a salt thereof, where each n is independently 1 to 20.

In still further embodiments, the compound is of the following structure:

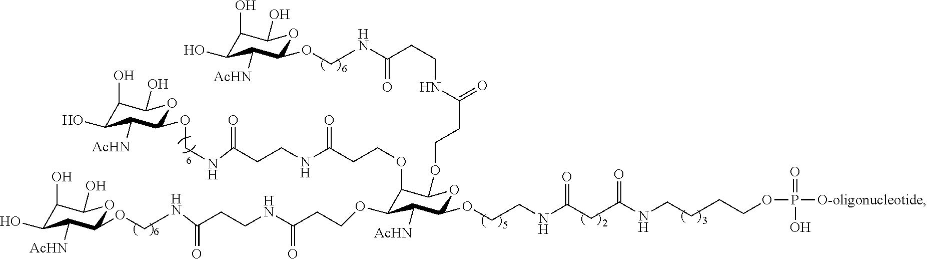

or a salt thereof.

In particular embodiments, the compound is of the following structure:

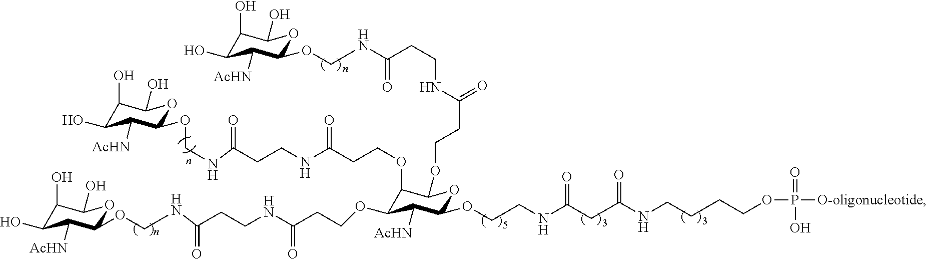

or a salt thereof,
where each n is independently 1 to 20.

In further embodiments, the compound is:

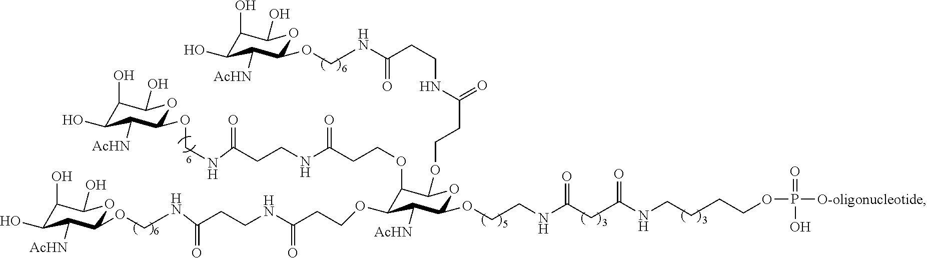

or a salt thereof.

In yet further embodiments, the compound is of the following structure:

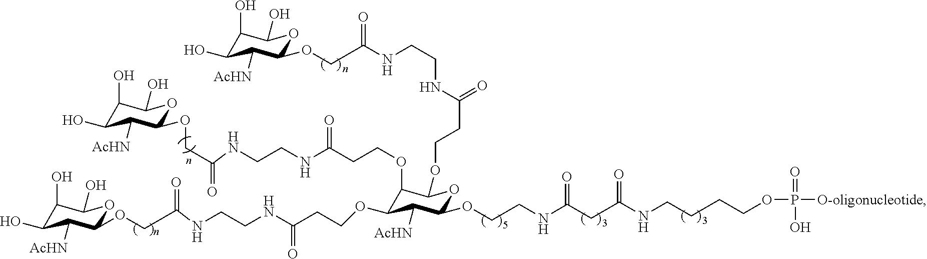

or a salt thereof,
where each n is independently 1 to 20.

In still further embodiments, the compound is:

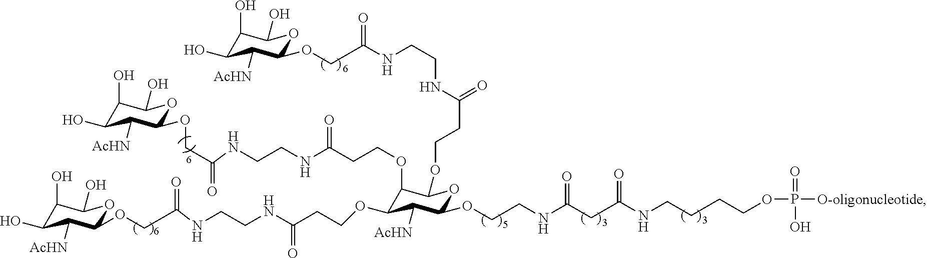

or a salt thereof.

In some embodiments, the compound is:

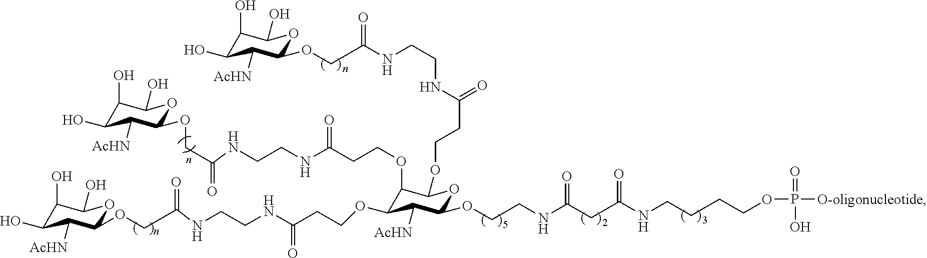

or a salt thereof, where each n is independently 1 to 20.

In certain embodiments, the compound is:

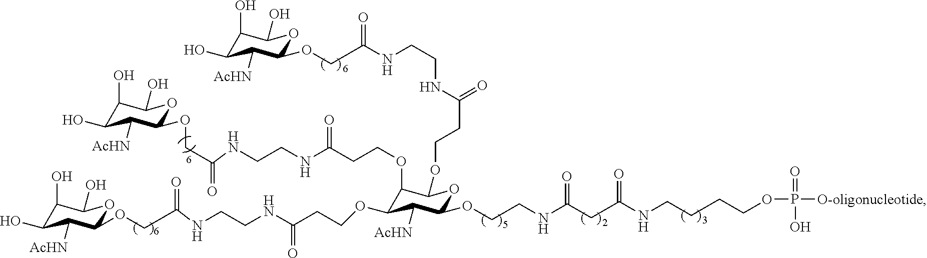

or a salt thereof.

In another aspect, the invention provides a method of delivering a therapeutically active agent to a cell having one or more surface receptors by contacting the cell with the compound of the invention, or a salt thereof, where at least one T is a ligand, and Z is a therapeutically active agent.

In some embodiments, the cell is in a tissue. In certain embodiments, the tissue is in a subject.

In yet another aspect, the invention provides a method of producing the compound of the invention, in which Z is a therapeutically active agent, by producing a product of a reaction between the compound of the invention, in which Z is a conjugation moiety and at least one T is a protected ligand, with a compound of formula (III):

Z¹—Z², tm (III)

or a salt thereof,
where
Z¹ is a complementary conjugation moiety; and
Z² is a therapeutically active agent.

In some embodiments, the method further includes deprotecting the product to produce the compound of the invention, in which Z is a therapeutically active agent and at least one T is a ligand.

### Definitions

Various terms used throughout the present description may be read and understood as follows, unless the context indicates otherwise: “or” as used throughout is inclusive, as though written “and/or”; singular articles and pronouns as used throughout include their plural forms, and vice versa; “exemplary” should be understood as “illustrative” or “exemplifying” and not necessarily as “preferred” over other embodiments. Further definitions for terms may be set out herein; these may apply to prior and subsequent instances of those terms, as will be understood from a reading of the present description.

The term “acyl,” as used herein, represents a chemical substituent of formula —C(O)—R, where R is alkyl, aryl, arylalkyl, cycloalkyl, heterocyclyl, heterocyclyl alkyl, heteroaryl, or heteroaryl alkyl. An optionally substituted acyl is an acyl that is optionally substituted as described herein for each group R.

The term “acyloxy,” as used herein, represents a chemical substituent of formula —OR, where R is acyl. An optionally substituted acyloxy is an acyloxy that is optionally substituted as described herein for acyl.

The term “alkane-tetrayl,” as used herein, represents a tetravalent, acyclic, straight or branched chain, saturated hydrocarbon group having from 1 to 16 carbons, unless otherwise specified. Alkane-tetrayl may be optionally substituted as described for alkyl.

The term “alkane-triyl,” as used herein, represents a trivalent, acyclic, straight or branched chain, saturated hydrocarbon group having from 1 to 16 carbons, unless otherwise specified. Alkane-triyl may be optionally substituted as described for alkyl.

The term “alkanoyl,” as used herein, represents a chemical substituent of formula —C(O)—R, where R is alkyl. An optionally substituted alkanoyl is an alkanoyl that is optionally substituted as described herein for alkyl.

The term “alkoxy,” as used herein, represents a chemical substituent of formula —OR, where R is a C₁₋₆ alkyl group, unless otherwise specified. An optionally substituted alkoxy is an alkoxy group that is optionally substituted as defined herein for alkyl.

The term “alkyl,” as used herein, refers to an acyclic straight or branched chain saturated hydrocarbon group, which, when unsubstituted, has from 1 to 12 carbons, unless otherwise specified. In certain preferred embodiments, unsubstituted alkyl has from 1 to 6 carbons. Alkyl groups are exemplified by methyl; ethyl; n- and iso-propyl; n-, sec-, iso- and tert-butyl; neopentyl, and the like, and may be optionally substituted, valency permitting, with one, two, three, or, in the case of alkyl groups of two carbons or more, four or more substituents independently selected from the group consisting of: alkoxy; acyloxy; amino; aryl; aryloxy; azido; cycloalkyl; cycloalkoxy; halo; heterocyclyl; heteroaryl; heterocyclylalkyl; heteroarylalkyl; heterocyclyloxy; heteroaryloxy; hydroxy; nitro; thiol; silyl; cyano; ═O; ═S; and ═NR′, where R′ is H, alkyl, aryl, or heterocyclyl. In some embodiments, a substituted alkyl includes two substituents (oxo and hydroxy, or oxo and alkoxy) to form a group -L-CO—R, where L is a bond or optionally substituted C₁₋₁₁ alkylene, and R is hydroxyl or alkoxy. Each of the substituents may itself be unsubstituted or, valency permitting, substituted with unsubstituted substituent(s) defined herein for each respective group.

The term “alkylene,” as used herein, represents a divalent substituent that is a monovalent alkyl having one hydrogen atom replaced with a valency. An optionally substituted alkylene is an alkylene that is optionally substituted as described herein for alkyl.

The term “aryl,” as used herein, represents a mono-, bicyclic, or multicyclic carbocyclic ring system having one or two aromatic rings. Aryl group may include from 6 to 10 carbon atoms. All atoms within an unsubstituted carbocyclic aryl group are carbon atoms. Non-limiting examples of carbocyclic aryl groups include phenyl, naphthyl, 1,2-dihydronaphthyl, 1,2,3,4-tetrahydronaphthyl, fluorenyl, indanyl, indenyl, etc. The aryl group may be unsubstituted or substituted with one, two, three, four, or five substituents independently selected from the group consisting of: alkyl; alkoxy; acyloxy; amino; aryl; aryloxy; azido; cycloalkyl; cycloalkoxy; halo; heterocyclyl; heteroaryl; heterocyclylalkyl; heteroarylalkyl; heterocyclyloxy; heteroaryloxy; hydroxy; nitro; thiol; silyl; and cyano. Each of the substituents may itself be unsubstituted or substituted with unsubstituted substituent(s) defined herein for each respective group.

The term “arylalkyl,” as used herein, represents an alkyl group substituted with an aryl group. The aryl and alkyl portions may be optionally substituted as the individual groups as described herein.

The term “arylene,” as used herein, represents a divalent substituent that is an aryl having one hydrogen atom replaced with a valency. An optionally substituted arylene is an arylene that is optionally substituted as described herein for aryl.

The term “aryloxy,” as used herein, represents a group —OR, where R is aryl. Aryloxy may be an optionally substituted aryloxy. An optionally substituted aryloxy is aryloxy that is optionally substituted as described herein for aryl.

The term “bicyclic sugar moiety,” as used herein, represents a modified sugar moiety including two fused rings. In certain embodiments, the bicyclic sugar moiety includes a furanosyl ring.

The expression “C_{x-y},” as used herein, indicates that the group, the name of which immediately follows the expression, when unsubstituted, contains a total of from x to y carbon atoms. If the group is a composite group (e.g., arylalkyl), C_{x-y} indicates that the portion, the name of which immediately follows the expression, when unsubstituted, contains a total of from x to y carbon atoms. For example, (C₆₋₁₀-aryl)-C₁₋₆-alkyl is a group, in which the aryl portion, when unsubstituted, contains a total of from 6 to 10 carbon atoms, and the alkyl portion, when unsubstituted, contains a total of from 1 to 6 carbon atoms.

The term “contiguous,” as used herein in the context of an oligonucleotide, refers to nucleosides, nucleobases, sugar moieties, or internucleoside linkages that are immediately adjacent to each other. For example, “contiguous nucleobases” means nucleobases that are immediately adjacent to each other in a sequence.

The term “cycloalkyl,” as used herein, refers to a cyclic alkyl group having from three to ten carbons (e.g., a C₃-C₁₀ cycloalkyl), unless otherwise specified. Cycloalkyl groups may be monocyclic or bicyclic. Bicyclic cycloalkyl groups may be of bicyclo[p.q.0]alkyl type, in which each of p and q is, independently, 1, 2, 3, 4, 5, 6, or 7, provided that the sum of p and q is 2, 3, 4, 5, 6, 7, or 8. Alternatively, bicyclic cycloalkyl groups may include bridged cycloalkyl structures, e.g., bicyclo[p.q.r]alkyl, in which r is 1, 2, or 3, each of p and q is, independently, 1, 2, 3, 4, 5, or 6, provided that the sum of p, q, and r is 3, 4, 5, 6, 7, or 8. The cycloalkyl group may be a spirocyclic group, e.g., spiro[p.q]alkyl, in which each of p and q is, independently, 2, 3, 4, 5, 6, or 7, provided that the sum of p and q is 4, 5, 6, 7, 8, or 9. Non-limiting examples of cycloalkyl include cyclopropyl, cyclobutyl, cyclopentyl, cyclohexyl, cycloheptyl, 1-bicyclo[2.2.1.]heptyl, 2-bicyclo[2.2.1.]heptyl, 5-bicyclo[2.2.1.]heptyl, 7-bicyclo[2.2.1.]heptyl, and decalinyl. The cycloalkyl group may be unsubstituted or substituted (e.g., optionally substituted cycloalkyl) with one, two, three, four, or five substituents independently selected from the group consisting of: alkyl; alkoxy; acyloxy; amino; aryl; aryloxy; azido; cycloalkyl; cycloalkoxy; halo; heterocyclyl; heteroaryl; heterocyclylalkyl; heteroarylalkyl; heterocyclyloxy; heteroaryloxy; hydroxy; nitro; thiol; silyl; cyano; ═O; ═S; ═NR′, where R′ is H, alkyl, aryl, or heterocyclyl. Each of the substituents may itself be unsubstituted or substituted with unsubstituted substituent(s) defined herein for each respective group.

The term “cycloalkylene,” as used herein, represents a divalent substituent that is a cycloalkyl having one hydrogen atom replaced with a valency. An optionally substituted cycloalkylene is a cycloalkylene that is optionally substituted as described herein for cycloalkyl.

The term “cycloalkoxy,” as used herein, represents a group —OR, where R is cycloalkyl. Cycloalkoxy may be an optionally substituted cycloalkoxy. An optionally substituted cycloalkoxy is cycloalkoxy that is optionally substituted as described herein for cycloalkyl.

The term “duplex,” as used herein, represents two oligonucleotides that are paired through hybridization of complementary nucleobases.

The term “halo,” as used herein, represents a halogen selected from bromine, chlorine, iodine, and fluorine.

The term “heteroalkyl,” as used herein, refers to an alkyl group interrupted one or more times by one or two heteroatoms each time. Each heteroatom is independently O, N, or S. None of the heteroalkyl groups includes two contiguous oxygen atoms. The heteroalkyl group may be unsubstituted or substituted (e.g., optionally substituted heteroalkyl). When heteroalkyl is substituted and the substituent is bonded to the heteroatom, the substituent is selected according to the nature and valency of the heteratom. Thus, the substituent bonded to the heteroatom, valency permitting, is selected from the group consisting of ═O, —N(R^{N2})₂, —SO₂OR^{N3}, —SO₂R^{N2}, —SOR^{N3}, —COOR^{N3}, an N protecting group, alkyl, aryl, cycloalkyl, heterocyclyl, or cyano, where each R^{N2} is independently H, alkyl, cycloalkyl, aryl, or heterocyclyl, and each R^{N3} is independently alkyl, cycloalkyl, aryl, or heterocyclyl. Each of these substituents may itself be unsubstituted or substituted with unsubstituted substituent(s) defined herein for each respective group. When heteroalkyl is substituted and the substituent is bonded to carbon, the substituent is selected from those described for alkyl, provided that the substituent on the carbon atom bonded to the heteroatom is not Cl, Br, or I. In some embodiments, carbon atoms are found at the termini of a heteroalkyl group. In some embodiments, heteroalkyl is PEG.

The term “heteroalkylene,” as used herein, represents a divalent substituent that is a heteroalkyl having one hydrogen atom replaced with a valency. An optionally substituted heteroalkylene is a heteroalkylene that is optionally substituted as described herein for heteroalkyl.

The term “heteroaryl,” as used herein, represents a monocyclic 5-, 6-, 7-, or 8-membered ring system, or a fused or bridging bicyclic, tricyclic, or tetracyclic ring system; the ring system contains one, two, three, or four heteroatoms independently selected from the group consisting of nitrogen, oxygen, and sulfur; and at least one of the rings is an aromatic ring. Non-limiting examples of heteroaryl groups include benzimidazolyl, benzofuryl, benzothiazolyl, benzothienyl, benzoxazolyl, furyl, imidazolyl, indolyl, isoindazolyl, isoquinolinyl, isothiazolyl, isothiazolyl, isoxazolyl, oxadiazolyl, oxazolyl, purinyl, pyrrolyl, pyridinyl, pyrazinyl, pyrimidinyl, qunazolinyl, quinolinyl, thiadiazolyl (e.g., 1,3,4-thiadiazole), thiazolyl, thienyl, triazolyl, tetrazolyl, dihydroindolyl, tetrahydroquinolyl, tetrahydroisoquinolyl, etc. The term bicyclic, tricyclic, and tetracyclic heteroaryls include at least one ring having at least one heteroatom as described above and at least one aromatic ring. For example, a ring having at least one heteroatom may be fused to one, two, or three carbocyclic rings, e.g., an aryl ring, a cyclohexane ring, a cyclohexene ring, a cyclopentane ring, a cyclopentene ring, or another monocyclic heterocyclic ring. Examples of fused heteroaryls include 1,2,3,5,8,8a-hexahydroindolizine; 2,3-dihydrobenzofuran; 2,3-dihydroindole; and 2,3-dihydrobenzothiophene. Heteroaryl may be optionally substituted with one, two, three, four, or five substituents independently selected from the group consisting of: alkyl; alkoxy; acyloxy; aryloxy; amino; arylalkoxy; cycloalkyl; cycloalkoxy; halogen; heterocyclyl; heterocyclyl alkyl; heteroaryl; heteroaryl alkyl; heterocyclyloxy; heteroaryloxy; hydroxyl; nitro; thiol; cyano; ═O; —NR₂, where each R is independently hydrogen, alkyl, acyl, aryl, arylalkyl, cycloalkyl, heterocyclyl, or heteroaryl; —COOR^{A}, where R^{A} is hydrogen, alkyl, aryl, arylalkyl, cycloalkyl, heterocyclyl, or heteroaryl; and —CON(R^{B})₂, where each R^{B} is independently hydrogen, alkyl, aryl, arylalkyl, cycloalkyl, heterocyclyl, or heteroaryl. Each of the substituents may itself be unsubstituted or substituted with unsubstituted substituent(s) defined herein for each respective group.

The term “heteroarylene,” as used herein, represents a divalent substituent that is a heteroaryl having one hydrogen atom replaced with a valency. An optionally substituted heteroarylene is a heteroarylene that is optionally substituted as described herein for heteroaryl.

The term “heteroaryloxy,” as used herein, refers to a structure —OR, in which R is heteroaryl. Heteroaryloxy can be optionally substituted as defined for heteroaryl.

The term “heterocyclyl,” as used herein, represents a monocyclic, bicyclic, tricyclic, or tetracyclic ring system having fused or bridging 4-, 5-, 6-, 7-, or 8-membered rings, unless otherwise specified, the ring system containing one, two, three, or four heteroatoms independently selected from the group consisting of nitrogen, oxygen, and sulfur. Heterocyclyl may be aromatic or non-aromatic. An aromatic heterocyclyl is heteroaryl as described herein. Non-aromatic 5-membered heterocyclyl has zero or one double bonds, non-aromatic 6- and 7-membered heterocyclyl groups have zero to two double bonds, and non-aromatic 8-membered heterocyclyl groups have zero to two double bonds and/or zero or one carbon-carbon triple bond. Heterocyclyl groups have a carbon count of 1 to 16 carbon atoms unless otherwise specified. Certain heterocyclyl groups may have a carbon count up to 9 carbon atoms. Non-aromatic heterocyclyl groups include pyrrolinyl, pyrrolidinyl, pyrazolinyl, pyrazolidinyl, imidazolinyl, imidazolidinyl, piperidinyl, homopiperidinyl, piperazinyl, pyridazinyl, oxazolidinyl, isoxazolidiniyl, morpholinyl, thiomorpholinyl, thiazolidinyl, isothiazolidinyl, thiazolidinyl, tetrahydrofuranyl, dihydrofuranyl, tetrahydrothienyl, dihydrothienyl, pyranyl, dihydropyranyl, dithiazolyl, etc. The term “heterocyclyl” also represents a heterocyclic compound having a bridged multicyclic structure in which one or more carbons and/or heteroatoms bridges two non-adjacent members of a monocyclic ring, e.g., quinuclidine, tropanes, or diaza-bicyclo[2.2.2]octane. The term “heterocyclyl” includes bicyclic, tricyclic, and tetracyclic groups in which any of the above heterocyclic rings is fused to one, two, or three carbocyclic rings, e.g., a cyclohexane ring, a cyclohexene ring, a cyclopentane ring, a cyclopentene ring, or another heterocyclic ring. Examples of fused heterocyclyls include 1,2,3,5,8,8a-hexahydroindolizine; 2,3-dihydrobenzofuran; 2,3-dihydroindole; and 2,3-dihydrobenzothiophene. The heterocyclyl group may be unsubstituted or substituted with one, two, three, four or five substituents independently selected from the group consisting of: alkyl; alkoxy; acyloxy; aryloxy; amino; arylalkoxy; cycloalkyl; cycloalkoxy; halogen; heterocyclyl; heterocyclyl alkyl; heteroaryl; heteroaryl alkyl; heterocyclyloxy; heteroaryloxy; hydroxyl; nitro; thiol; cyano; ═O; ═S; —NR₂, where each R is independently hydrogen, alkyl, acyl, aryl, arylalkyl, cycloalkyl, heterocyclyl, or heteroaryl; —COOR^{A}, where R^{A} is hydrogen, alkyl, aryl, arylalkyl, cycloalkyl, heterocyclyl, or heteroaryl; and —CON(R^{B})₂, where each R^{B} is independently hydrogen, alkyl, aryl, arylalkyl, cycloalkyl, heterocyclyl, or heteroaryl.

The term “heterocyclyl alkyl,” as used herein, represents an alkyl group substituted with a heterocyclyl group. The heterocyclyl and alkyl portions of an optionally substituted heterocyclyl alkyl are optionally substituted as described for heterocyclyl and alkyl, respectively.

The term “heterocyclylene,” as used herein, represents a divalent substituent that is a heterocyclyl having one hydrogen atom replaced with a valency. An optionally substituted heterocyclylene is a heterocyclylene that is optionally substituted as described herein for heterocyclyl.

The term “heterocyclyloxy,” as used herein, refers to a structure —OR, in which R is heterocyclyl. Heterocyclyloxy can be optionally substituted as described for heterocyclyl.

The term “heteroorganic,” as used herein, refers to (i) an acyclic hydrocarbon interrupted one or more times by one or two heteroatoms each time, or (ii) a cyclic hydrocarbon including one or more (e.g., one, two, three, or four) endocyclic heteroatoms. Each heteroatom is independently O, N, or S. None of the heteroorganic groups includes two contiguous oxygen atoms. An optionally substituted heteroorganic group is a heteroorganic group that is optionally substituted as described herein for alkyl.

The term “hydrocarbon,” as used herein, refers to an acyclic, branched or acyclic, linear compound or group, or a monocyclic, bicyclic, tricyclic, or tetracyclic compound or group. The hydrocarbon, when unsubstituted, consists of carbon and hydrogen atoms. Unless specified otherwise, an unsubstituted hydrocarbon includes a total of 1 to 60 carbon atoms (e.g., 1 to 16, 1 to 12, or 1 to 6 carbon atoms). An optionally substituted hydrocarbon is an optionally substituted acyclic hydrocarbon or an optionally substituted cyclic hydrocarbon. An optionally substituted acyclic hydrocarbon is optionally substituted as described herein for alkyl. An optionally substituted cyclic hydrocarbon is an optionally substituted aromatic hydrocarbon or an optionally substituted non-aromatic hydrocarbon. An optionally substituted aromatic hydrocarbon is optionally substituted as described herein for aryl. An optionally substituted non-aromatic cyclic hydrocarbon is optionally substituted as described herein for cycloalkyl. In some embodiments, an acyclic hydrocarbon is alkyl, alkylene, alkane-triyl, or alkane-tetrayl. In certain embodiments, a cyclic hydrocarbon is aryl or arylene. In particular embodiments, a cyclic hydrocarbon is cycloalkyl or cycloalkylene.

The terms “hydroxyl” and “hydroxy,” as used interchangeably herein, represent —OH.

The term “hydrophobic moiety,” as used herein, represents a monovalent group covalently linked to an oligonucleotide backbone, where the monovalent group is a bile acid (e.g., cholic acid, taurocholic acid, deoxycholic acid, oleyl lithocholic acid, or oleoyl cholenic acid), glycolipid, phospholipid, sphingolipid, isoprenoid, vitamin, saturated fatty acid, unsaturated fatty acid, fatty acid ester, triglyceride, pyrene, porphyrine, texaphyrine, adamantine, acridine, biotin, coumarin, fluorescein, rhodamine, Texas-Red, digoxygenin, dimethoxytrityl, t-butydimethylsilyl, t-butyldiphenylsilyl, cyanine dye (e.g., Cy3 or Cy5), Hoechst 33258 dye, psoralen, or ibuprofen. Non-limiting examples of the monovalent group include ergosterol, stigmasterol, β-sitosterol, campesterol, fucosterol, saringosterol, avenasterol, coprostanol, cholesterol, vitamin A, vitamin D, vitamin E, cardiolipin, and carotenoids. The linker connecting the monovalent group to the oligonucleotide may be an optionally substituted C₁₋₆₀ hydrocarbon (e.g., optionally substituted C₁₋₆₀ alkylene) or an optionally substituted C₂₋₆₀ heteroorganic (e.g., optionally substituted C₂₋₆₀ heteroalkylene), where the linker may be optionally interrupted with one, two, or three instances independently selected from the group consisting of an optionally substituted arylene, optionally substituted heterocyclylene, and optionally substituted cycloalkylene. The linker may be bonded to an oligonucleotide through, e.g., an oxygen atom attached to a 5′-terminal carbon atom, a 3′-terminal carbon atom, a 5′-terminal phosphate or phosphorothioate, a 3′-terminal phosphate or phosphorothioate, or an internucleoside linkage.

The term “internucleoside linkage,” as used herein, represents a divalent group or covalent bond that forms a covalent linkage between adjacent nucleosides in an oligonucleotide. An internucleoside linkage is an unmodified internucleoside linkage or a modified internucleoside linkage. An “unmodified internucleoside linkage” is a phosphate (—O—P(O)(OH)—O—) internucleoside linkage (“phosphate phosphodiester”). A “modified internucleoside linkage” is an internucleoside linkage other than a phosphate phosphodiester. The two main classes of modified internucleoside linkages are defined by the presence or absence of a phosphorus atom. Non-limiting examples of phosphorus-containing internucleoside linkages include phosphodiester linkages, phosphotriester linkages, phosphorothioate diester linkages, phosphorothioate triester linkages, phosphorodithioate linkages, boranophosphonate linkages, morpholino internucleoside linkages, methylphosphonates, and phosphoramidate. Non-limiting examples of non-phosphorus internucleoside linkages include methylenemethylimino (—CH₂—N(CH₃)—O—CH₂—), thiodiester (—O—C(O)—S—), thionocarbamate (—O—C(O)(NH)—S—), siloxane (—O—Si(H)₂—O—), and N,N′-dimethylhydrazine (—CH₂—N(CH₃)—N(CH₃)—). Phosphorothioate linkages are phosphodiester linkages and phosphotriester linkages in which one of the non-bridging oxygen atoms is replaced with a sulfur atom. In some embodiments, an internucleoside linkage is a group of the following structure:

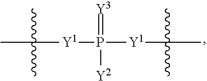

where
X is a monosaccharide;
each Y¹ is independently —O—, —S—, —N(-L-R¹)—, or L;
Y² is (T-L¹-)ₚ-X-L²- or R¹-L-Y¹—;
Y³ is O, S, B, or Se;
each L is independently a covalent bond or a covalent linker (e.g., optionally substituted C₁₋₆₀ hydrocarbon linker or optionally substituted C₂₋₆₀ heteroorganic linker);
each L¹ is independently a covalent linker;
L² is a conjugation linker;
each R¹ is independently hydrogen, —S—S—R², —O—CO—R², —S—CO—R², optionally substituted C₁₋₉ heterocyclyl, or a hydrophobic moiety; and
each R² is independently optionally substituted C₁₋₁₀ alkyl, optionally substituted C₂₋₁₀ heteroalkyl, optionally substituted C₆₋₁₀ aryl, optionally substituted C₆₋₁₀ aryl C₁₋₆ alkyl, optionally substituted C₁₋₉ heterocyclyl, or optionally substituted C₁₋₉ heterocyclyl C₁₋₆ alkyl;
p is 1 to 5;
each T is independently a ligand or a protected ligand.

When L is a covalent bond, R¹ is hydrogen, Y³ is oxygen, all Y¹ and groups are —O—, and L is a bond, the internucleoside group is known as a phosphate phosphodiester. When L is a covalent bond, R¹ is hydrogen, Y³ is sulfur, all Y¹ groups are —O—, and L is a bond, the internucleoside group is known as a phosphorothioate diester. When Y³ is oxygen, all Y¹ groups are —O—, and either (1) Y² is (T-L¹-)ₚ-X-L²- or (2) R¹-L-Y¹-, in which L is a linker or R¹ is not a hydrogen, the internucleoside group is known as a phosphotriester. When Y³ is sulfur, all Y¹ groups are —O—, and either (1) Y² is (T-L¹-)ₚ-X-L²- or (2) R¹-L-Y¹-, in which L is a linker or R¹ is not a hydrogen, the internucleoside group is known as a phosphorothioate triester.

The term “morpholino,” as used herein in reference to a class of oligonucleotides, represents an oligomer of at least 10 morpholino monomer units interconnected by morpholino internucleoside linkages. A morpholino includes a 5′ group and a 3′ group. For example, a morpholino may be of the following structure:

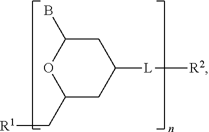

where
n is at least 10 (e.g., 12 to 50) indicating the number of morpholino units;
each B is independently a nucleobase;
R¹ is a 5′ group;
R² is a 3′ group; and
L is (i) a morpholino internucleoside linkage or, (ii) if L is attached to R², a covalent bond.

A 5′ group in morpholino may be, e.g., hydroxyl, a hydrophobic moiety, phosphate, diphosphate, triphosphate, phosphorothioate, diphosphorothioate, triphosphorothioate, phosphorodithioate, disphorodithioate, triphosphorodithioate, phosphonate, phosphoramidate, a cell penetrating peptide, an endosomal escape moiety, or a neutral organic polymer. A 3′ group in morpholino may be, e.g., hydrogen, a hydrophobic moiety, phosphate, diphosphate, triphosphate, phosphorothioate, diphosphorothioate, triphosphorothioate, phosphorodithioate, disphorodithioate, triphosphorodithioate, phosphonate, phosphoramidate, a cell penetrating peptide, an endosomal escape moiety, or a neutral organic polymer.

The term “morpholino internucleoside linkage,” as used herein, represents a divalent group of the following structure:

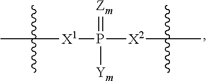

where
Zₘ is O or S;
X¹ is a bond, —CH₂—, or —O—;
X² is a bond, —CH₂—O—, or —O—; and
Yₘ is —NR₂, where each R is independently C₁₋₆ alkyl (e.g., methyl), or both R combine together with the nitrogen atom to which they are attached to form a C₂₋₉ heterocyclyl (e.g., N-piperazinyl);
   provided that both X¹ and X² are not simultaneously a bond.

The term “nucleobase,” as used herein, represents a nitrogen-containing heterocyclic ring found at the 1′ position of the ribofuranose/2′-deoxyribofuranose of a nucleoside. Nucleobases are unmodified or modified. As used herein, “unmodified” or “natural” nucleobases include the purine bases adenine (A) and guanine (G), and the pyrimidine bases thymine (T), cytosine (C), and uracil (U). Modified nucleobases include 5-substituted pyrimidines, 6-azapyrimidines, alkyl or alkynyl substituted pyrimidines, alkyl substituted purines, and N-2, N-6 and O-6 substituted purines, as well as synthetic and natural nucleobases, e.g., 5-methylcytosine, 5-hydroxymethyl cytosine, xanthine, hypoxanthine, 2-aminoadenine, 6-alkyl (e.g., 6-methyl) adenine and guanine, 2-alkyl (e.g., 2-propyl) adenine and guanine, 2-thiouracil, 2-thiothymine, 2-thiocytosine, 5-halouracil, 5-halocytosine, 5-propynyl uracil, 5-propynyl cytosine, 5-trifluoromethyl uracil, 5-trifluoromethyl cytosine, 7-methyl guanine, 7-methyl adenine, 8-azaguanine, 8-azaadenine, 7-deazaguanine, 7-deazaadenine, 3-deazaguanine, 3-deazaadenine. Certain nucleobases are particularly useful for increasing the binding affinity of nucleic acids, e g., 5-substituted pyrimidines; 6-azapyrimidines; N2-, N6-, and/or O6-substituted purines. Nucleic acid duplex stability can be enhanced using, e.g., 5-methylcytosine. Non-limiting examples of nucleobases include: 2-aminopropyladenine, 5-hydroxymethyl cytosine, xanthine, hypoxanthine, 2-aminoadenine, 6-N-methylguanine, 6-N-methyladenine, 2-propyladenine, 2-thiouracil, 2-thiothymine and 2-thiocytosine, 5-propynyl (—C≡C—CH₃) uracil, 5-propynylcytosine, 6-azouracil, 6-azocytosine, 6-azothymine, 5-ribosyluracil (pseudouracil), 4-thiouracil, 8-halo, 8-amino, 8-thiol, 8-thioalkyl, 8-hydroxyl, 8-aza and other 8-substituted purines, 5-halo, particularly 5-bromo, 5-trifluoromethyl, 5-halouracil, and 5-halocytosine, 7-methylguanine, 7-methyladenine, 2-F-adenine, 2-aminoadenine, 7-deazaguanine, 7-deazaadenine, 3-deazaguanine, 3-deazaadenine, 6-N-benzoyladenine, 2-N-isobutyrylguanine, 4-N-benzoylcytosine, 4-N-benzoyluracil, 5-methyl 4-N-benzoylcytosine, 5-methyl 4-N-benzoyluracil, universal bases, hydrophobic bases, promiscuous bases, size-expanded bases, and fluorinated bases. Further modified nucleobases include tricyclic pyrimidines, such as 1,3-diazaphenoxazine-2-one, 1,3-diazaphenothiazine-2-one and 9-(2-aminoethoxy)-1,3-diazaphenoxazine-2-one (G-clamp). Modified nucleobases may also include those in which the purine or pyrimidine base is replaced with other heterocycles, for example, 7-deazaadenine, 7-deazaguanine, 2-aminopyridine, or 2-pyridone. Further nucleobases include those disclosed in U.S. Pat. No. 3,687,808; The Concise Encyclopedia of Polymer Science and Engineering, Kroschwitz, J. I., Ed., John Wiley & Sons, 1990, 858-859; Englisch et al., Angewandte Chemie, International Edition, 1991, 30, 613; Sanghvi, Y. S., Chapter 15, Antisense Research and Applications, Crooke, S. T. and Lebleu, B., Eds., CRC Press, 1993, 273-288; and in Chapters 6 and 15, Antisense Drug Technology, Crooke S. T., Ed., CRC Press, 2008, 163-166 and 442-443.

The term “nucleoside,” as used herein, represents sugar-nucleobase compounds and groups known in the art (e.g., modified or unmodified ribofuranose-nucleobase and 2′-deoxyribofuranose-nucleobase compounds and groups known in the art). The sugar may be ribofuranose. The sugar may be modified or unmodified. An unmodified sugar nucleoside is ribofuranose or 2′-deoxyribofuranose having an anomeric carbon bonded to a nucleobase. An unmodified nucleoside is ribofuranose or 2′-deoxyribofuranose having an anomeric carbon bonded to an unmodified nucleobase. Non-limiting examples of unmodified nucleosides include adenosine, cytidine, guanosine, uridine, 2′-deoxyadenosine, 2′-deoxycytidine, 2′-deoxyguanosine, and thymidine. The modified compounds and groups include one or more modifications selected from the group consisting of nucleobase modifications and sugar modifications described herein. A nucleobase modification is a replacement of an unmodified nucleobase with a modified nucleobase. A sugar modification may be, e.g., a 2′-substitution, locking, carbocyclization, or unlocking. A 2′-substitution is a replacement of 2′-hydroxyl in ribofuranose with 2′-fluoro, 2′-methoxy, or 2′-(2-methoxy)ethoxy. A locking modification is an incorporation of a bridge between 4′-carbon atom and 2′-carbon atom of ribofuranose. Nucleosides having a locking modification are known in the art as bridged nucleic acids, e.g., locked nucleic acids (LNA), ethylene-bridged nucleic acids (ENA), and cEt nucleic acids. The bridged nucleic acids are typically used as affinity enhancing nucleosides.

The term “nucleotide,” as used herein, represents a nucleoside bonded to an internucleoside linkage or a monovalent group of the following structure —X¹—P(X²)(R¹)₂, where X¹ is O, S, or NH, and X² is absent, ═O, or ═S, and each R¹ is independently —OH, —N(R²)₂, or —O—CH₂CH₂CN, where each R² is independently an optionally substituted alkyl, or both R² groups, together with the nitrogen atom to which they are attached, combine to form an optionally substituted heterocyclyl.

The term “oligonucleotide,” as used herein, represents a structure containing 10 or more (e.g., 10 to 50) contiguous nucleosides covalently bound together by internucleoside linkages. An oligonucleotide includes a 5′ end and a 3′ end. The 5′ end of an oligonucleotide may be, e.g., hydroxyl, a targeting moiety, a hydrophobic moiety, 5′ cap, phosphate, diphosphate, triphosphate, phosphorothioate, diphosphorothioate, triphosphorothioate, phosphorodithioate, diphosphrodithioate, triphosphorodithioate, phosphonate, phosphoramidate, a cell penetrating peptide, an endosomal escape moiety, or a neutral organic polymer. The 3′ end of an oligonucleotide may be, e.g., hydroxyl, a targeting moiety, a hydrophobic moiety, phosphate, diphosphate, tri phosphate, phosphorothioate, diphosphorothioate, triphosphorothioate, phosphorodithioate, disphorodithioate, triphosphorodithioate, phosphonate, phosphoramidate, a cell penetrating peptide, an endosomal escape moiety, or a neutral organic polymer (e.g., polyethylene glycol). An oligonucleotide having a 5′-hydroxyl or 5′-phosphate has an unmodified 5′ terminus. An oligonucleotide having a 5′ terminus other than 5′-hydroxyl or 5′-phosphate has a modified 5′ terminus. An oligonucleotide having a 3′-hydroxyl or 3′-phosphate has an unmodified 3′ terminus. An oligonucleotide having a 3′ terminus other than 3′-hydroxyl or 3′-phosphate has a modified 3′ terminus.

The term “oxo,” as used herein, represents a divalent oxygen atom (e.g., the structure of oxo may be shown as ═O).

The term “pharmaceutically acceptable,” as used herein, refers to those compounds, materials, compositions, and/or dosage forms, which are suitable for contact with the tissues of an individual (e.g., a human), without excessive toxicity, irritation, allergic response and other problem complications commensurate with a reasonable benefit/risk ratio.

The term “protecting group,” as used herein, represents a group intended to protect a functional group (e.g., a hydroxyl, an amino, or a carbonyl) from participating in one or more undesirable reactions during chemical synthesis. The term “O-protecting group,” as used herein, represents a group intended to protect an oxygen containing (e.g., phenol, hydroxyl or carbonyl) group from participating in one or more undesirable reactions during chemical synthesis. The term “N-protecting group,” as used herein, represents a group intended to protect a nitrogen containing (e.g., an amino or hydrazine) group from participating in one or more undesirable reactions during chemical synthesis. Commonly used O- and N-protecting groups are disclosed in Wuts, “Greene's Protective Groups in Organic Synthesis,” 4th Edition (John Wiley & Sons, New York, 2006), which is incorporated herein by reference. Exemplary O- and N-protecting groups include alkanoyl, aryloyl, or carbamyl groups such as formyl, acetyl, propionyl, pivaloyl, t-butylacetyl, 2-chloroacetyl, 2-bromoacetyl, trifluoroacetyl, trichloroacetyl, phthalyl, o-nitrophenoxyacetyl, α-chlorobutyryl, benzoyl, 4-chlorobenzoyl, 4-bromobenzoyl, t-butyldimethylsilyl, tri-iso-propylsilyloxymethyl, 4,4′-dimethoxytrityl, isobutyryl, phenoxyacetyl, 4-isopropylpehenoxyacetyl, dimethylformamidino, and 4-nitrobenzoyl.

Exemplary O-protecting groups for protecting carbonyl containing groups include, but are not limited to: acetals, acylals, 1,3-dithianes, 1,3-dioxanes, 1,3-dioxolanes, and 1,3-dithiolanes.

Other O-protecting groups include, but are not limited to: substituted alkyl, aryl, and arylalkyl ethers (e.g., trityl; methylthiomethyl; methoxymethyl; benzyloxymethyl; siloxymethyl; 2,2,2,-trichloroethoxymethyl; tetrahydropyranyl; tetrahydrofuranyl; ethoxyethyl; 1-[2-(trimethylsilyl)ethoxy]ethyl; 2-trimethylsilylethyl; t-butyl ether; p-chlorophenyl, p-methoxyphenyl, p-nitrophenyl, benzyl, p-methoxybenzyl, and nitrobenzyl); silyl ethers (e.g., trimethylsilyl; triethylsilyl; triisopropylsilyl; dimethylisopropylsilyl; t-butyldimethylsilyl; t-butyldiphenylsilyl; tribenzylsilyl; triphenylsilyl; and diphenymethylsilyl); carbonates (e.g., methyl, methoxymethyl, 9-fluorenylmethyl; ethyl; 2,2,2-trichloroethyl; 2-(trimethylsilyl)ethyl; vinyl, allyl, nitrophenyl; benzyl; methoxybenzyl; 3,4-dimethoxybenzyl; and nitrobenzyl).

Other N-protecting groups include, but are not limited to, chiral auxiliaries such as protected or unprotected D, L or D, L-amino acids such as alanine, leucine, phenylalanine, and the like; sulfonyl-containing groups such as benzenesulfonyl, p-toluenesulfonyl, and the like; carbamate forming groups such as benzyloxycarbonyl, p-chlorobenzyloxycarbonyl, p-methoxybenzyloxycarbonyl, p-nitrobenzyloxycarbonyl, 2-nitrobenzyloxycarbonyl, p-bromobenzyloxycarbonyl, 3,4-dimethoxybenzyloxycarbonyl, 3,5-dimethoxybenzyl oxycarbonyl, 2,4-dimethoxybenzyloxycarbonyl, 4-methoxybenzyloxycarbonyl, 2-nitro-4,5-dimethoxybenzyloxycarbonyl, 3,4,5-trimethoxybenzyloxycarbonyl, 1-(p-biphenylyl)-1-methylethoxycarbonyl, α,α-dimethyl-3,5-dimethoxybenzyloxycarbonyl, benzhydroxy carbonyl, t-butyloxycarbonyl, diisopropylmethoxycarbonyl, isopropoxycarbonyl, ethoxycarbonyl, methoxycarbonyl, allyloxycarbonyl, 2,2,2-trichloroethoxycarbonyl, phenoxycarbonyl, 4-nitrophenoxy carbonyl, fluorenyl-9-methoxycarbonyl, cyclopentyloxycarbonyl, adamantyloxycarbonyl, cyclohexyloxycarbonyl, phenylthiocarbonyl, and the like, arylalkyl groups such as benzyl, triphenylmethyl, benzyloxymethyl, and the like and silyl groups such as trimethylsilyl, and the like.

The term “pyrid-2-yl hydrazone,” as used herein, represents a group of the structure:

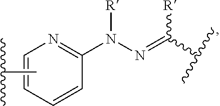

where each R′ is independently H or optionally substituted C₁₋₆ alkyl. Pyrid-2-yl hydrazone may be unsubstituted (i.e., each R′ is H).

GC

The term “stereochemically enriched,” as used herein, refers to a local stereochemical preference for one enantiomer of the recited group over the opposite enantiomer of the same group. Thus, an oligonucleotide containing a stereochemically enriched internucleoside linkage is an oligonucleotide, in which a stereogenic internucleoside linkage (e.g., phosphorothioate) of predetermined stereochemistry is present in preference to a stereogenic internucleoside linkage (e.g., phosphorothioate) of stereochemistry that is opposite of the predetermined stereochemistry. This preference can be expressed numerically using a diastereomeric ratio for the stereogenic internucleoside linkage (e.g., phosphorothioate) of the predetermined stereochemistry. The diastereomeric ratio for the stereogenic internucleoside linkage (e.g., phosphorothioate) of the predetermined stereochemistry is the molar ratio of the diastereomers having the identified stereogenic internucleoside linkage (e.g., phosphorothioate) with the predetermined stereochemistry relative to the diastereomers having the identified stereogenic internucleoside linkage (e.g., phosphorothioate) with the stereochemistry that is opposite of the predetermined stereochemistry. The diastereomeric ratio for the phosphorothioate of the predetermined stereochemistry may be greater than or equal to 1.1 (e.g., greater than or equal to 4, greater than or equal to 9, greater than or equal to 19, or greater than or equal to 39).

The term “subject,” as used herein, represents a human or non-human animal (e.g., a mammal) that is suffering from, or is at risk of, disease, disorder, or condition, as determined by a qualified professional (e.g., a doctor or a nurse practitioner) with or without known in the art laboratory test(s) of sample(s) from the subject.

A “sugar” or “sugar moiety,” includes naturally occurring sugars having a furanose ring or a structure that is capable of replacing the furanose ring of a nucleoside. Sugars included in the nucleosides of the invention may be non-furanose (or 4′-substituted furanose) rings or ring systems or open systems. Such structures include simple changes relative to the natural furanose ring (e.g., a six-membered ring). Alternative sugars may also include sugar surrogates wherein the furanose ring has been replaced with another ring system such as, e.g., a morpholino or hexitol ring system. Non-limiting examples of sugar moieties useful that may be included in the oligonucleotides of the invention include β-D-ribose, β-D-2′-deoxyribose, substituted sugars (e.g., 2′, 5′, and bis substituted sugars), 4′-S-sugars (e.g., 4′-S-ribose, 4′-S-2′-deoxyribose, and 4′-S-2′-substituted ribose), bicyclic sugar moieties (e.g., the 2′-O—CH₂-4′ or 2′-O—(CH₂)₂-4′ bridged ribose derived bicyclic sugars) and sugar surrogates (when the ribose ring has been replaced with a morpholino or a hexitol ring system).

The term “targeting moiety,” as used herein, represents a moiety (e.g., N-acetylgalactosamine cluster) that specifically binds or reactively associates or complexes with a receptor or other receptive moiety associated with a given target cell population. The targeting moiety included in the compounds of the invention is Yₚ—X-L²- as described herein, where p indicates the number of groups Y directly bonded to group X. An oligonucleotide including a targeting moiety is also referred to herein as a conjugate. A targeting moiety may include one or more ligands (e.g., 1 to 9 ligands, 1 to 6 ligands, 1 to 3 ligands, or 1 ligand). The ligand can be an antibody or an antigen-binding fragment or an engineered derivative thereof (e.g., Fcab or a fusion protein (e.g., scFv)). Alternatively, the ligand may be a small molecule (e.g., N-acetylgalactosamine). The ligand may target a cell expressing asialoglycoprotein receptor (ASGP-R), IgA receptor, HDL receptor, LDL receptor, or transferrin receptor. Non-limiting examples of the ligands include N-acetylgalactosamine, glycyrrhetinic acid, glycyrrhizin, lactobionic acid, lactoferrin, IgA, or a bile acid (e.g., lithocholyltaurine or taurocholic acid).

The term “therapeutically active agent,” as used herein, represents compounds and compound classes known as being therapeutically active. For example, a therapeutically active agent may be a therapeutically active oligonucleotide, e.g., an antisense oligonucleotide, splice-switching oligonucleotide, siRNA, miRNA, or CpG ODN.

The term “thiocarbonyl,” as used herein, represents a C(═S) group. Non-limiting example of functional groups containing a “thiocarbonyl” includes thioesters, thioketones, thioaldehydes, thioanhydrides, thioacyl chlorides, thioamides, thiocarboxylic acids, and thiocarboxylates.

The term “thioheterocyclylene,” as used herein, represents a divalent group —S—R′—, where R′ is a heterocyclylene as defined herein.

The term “thiol,” as used herein, represents an —SH group.

The term “triazolocycloalkenylene,” as used herein, refers to the heterocyclylenes containing a 1,2,3-triazole ring fused to an 8-membered ring, all of the endocyclic atoms of which are carbon atoms, and bridgehead atoms are sp²-hybridized carbon atoms. Triazocycloalkenylenes can be optionally substituted in a manner described for heterocyclyl.

The term “triazoloheterocyclylene,” as used herein, refers to the heterocyclylenes containing a 1,2,3-triazole ring fused to an 8-membered ring containing at least one heteroatom. The bridgehead atoms in triazoloheterocyclylene are carbon atoms. Triazoloheterocyclylenes can be optionally substituted in a manner described for heterocyclyl.

Enumeration of positions within oligonucleotides and nucleic acids, as used herein and unless specified otherwise, starts with the 5′-terminal nucleoside as 1 and proceeds in the 3′-direction.

The compounds described herein, unless otherwise noted, encompass isotopically enriched compounds (e.g., deuterated compounds), tautomers, and all stereoisomers and conformers (e.g. enantiomers, diastereomers, EIZ isomers, atropisomers, etc.), as well as racemates thereof and mixtures of different proportions of enantiomers or diastereomers, or mixtures of any of the foregoing forms as well as salts (e.g., pharmaceutically acceptable salts).

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### DETAILED DESCRIPTION

In general, the invention provides compounds that may be useful for targeting cells, e.g., in a tissue, e.g., in a subject. The compounds of the invention include a targeting moiety of the following structure:

Yₚ—X-L²-,

where
p is 1 to 5;
X is a monosaccharide;
each Y is independently -L¹-T, H, protecting group, optionally substituted hydrocarbon, or optionally substituted heteroorganic group, where each T is independently a ligand or a protected ligand, and each L¹ is independently a covalent linker; and
L² is a conjugation linker.

The compound of the invention may be a compound of formula (I):

Yₚ—X-L²-Z, (I)

or a salt thereof,
where
p is 1 to 5;
X is a monosaccharide;
each Y is independently -L¹-T, H, protecting group, optionally substituted hydrocarbon, or optionally substituted heteroorganic group, where each T is independently a ligand or a protected ligand, and each L¹ is independently a covalent linker;
L² is a conjugation linker; and
Z is a therapeutically active agent, protecting group, or a conjugation moiety.

In some embodiments, at least one Y is -L¹-T.

The monosaccharide may be N-acetylgalactosamine, galactosamine, galactose, mannose, allose, altrose, glucose, gulose, idose, talose, arabinose, lyxose, ribose, or xylose.

The group -L²-Z may be a group of the following structure:

-Q¹-Q²-Z,

where
Q¹ is [-Q³-Q⁴-Q⁵]ₛ-Q^{C}-B¹, where B¹ is a bond to Q²;
Q² is [-Q³-Q⁴-Q⁵]ₛB², where B² is a bond to Z;
each Q³ is independently absent, —CO—, —NH—, —O—, —S—, —SO₂—, —OC(O)—, —C(O)O—, —NHC(O)—, —C(O)NH—, —CH₂—, —CH₂NH—, —NHCH₂—, —CH₂O—, or —OCH₂—;
each Q⁴ is independently absent, optionally substituted C₁₋₁₂ alkylene, optionally substituted C₂₋₁₂ alkenylene, optionally substituted C₂₋₁₂ alkynylene, optionally substituted C₂₋₁₂ heteroalkylene, optionally substituted C₆₋₁₀ arylene, optionally substituted C₁₋₉ heteroarylene, or optionally substituted C₁₋₉ heterocyclylene;
each Q⁵ is independently absent, —CO—, —NH—, —O—, —S—, —SO₂—, —CH₂—, —C(O)O—, —OC(O)—, —C(O)NH—, —NHC(O)—, —NH—CH(R^{a})—C(O)—, —C(O)—CH(R^{a})—NH—, —OP(O)(OH)O—, or —OP(S)(OH)O—, where each R^{a} is independently H or optionally substituted C₁₋₁₂ alkyl;
Q^{C} is optionally substituted C₂₋₁₂ alkylene, optionally substituted C₂₋₁₂ heteroalkylene (e.g., a heteroalkylene containing —C(O)—N(H)—, —N(H)—C(O)—, —S(O)₂N(H)—, —N(H)—S(O)₂—, or —S—S—), optionally substituted C₁₋₁₂ thioheterocyclylene (e.g.,

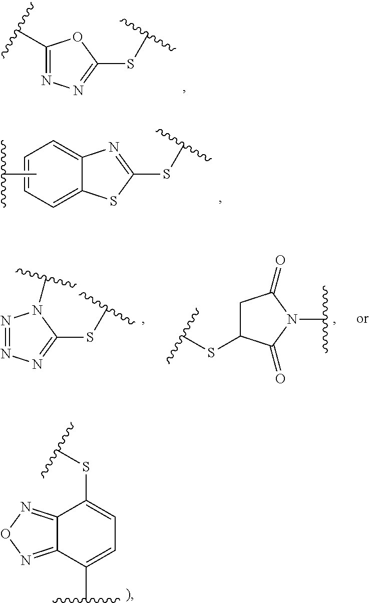

optionally substituted C₁₋₁₂ heterocyclylene (e.g., 1,2,3-triazole-1,4-diyl or

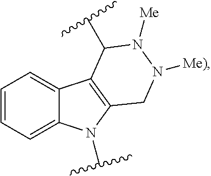

cyclobut-3-ene-1,2-dione-3,4-diyl, pyrid-2-yl hydrazone, optionally substituted C₆₋₁₆ triazoloheterocyclylene (e.g.,

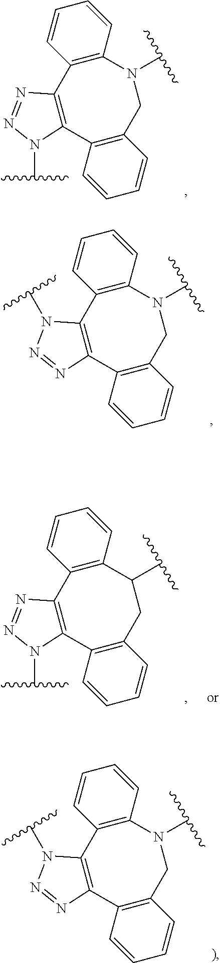

optionally substituted C₈₋₁₆ triazolocycloalkenylene

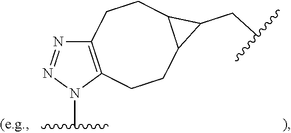 [SMILES: *CC1C2CCc3nnn(*)c3CCC12]

or a dihydropyridazine group (e.g., trans-

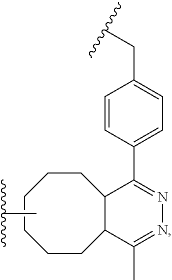

trans-

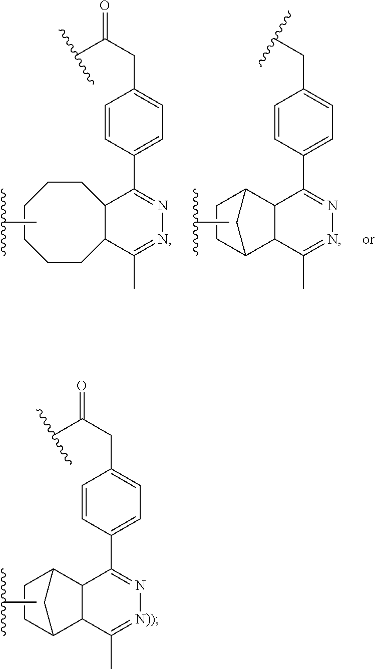

and
each s is independently 0 to 20.

In particular preferred embodiments, each Q⁵ is independently absent, —CO—, —NH—, —O—, —S—, —SO₂—, —CH₂—, —C(O)O—, —OC(O)—, —C(O)NH—, —NHC(O)—, —OP(O)(OH)O—, or —OP(S)(OH)O—.

The group -L²-Z may be a group of the following structure:

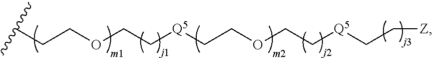

where
each of m1 and m2 is independently 0, 1, 2, 3, 4, 5, 6, 7, 8, 9, or 10; and
each of j1, j2, and j3 is independently 1, 2, 3, 4, or 5.

The group -L²-Z may be a group of the following structure:

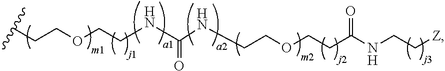

where a1 is 0 and a2 is 1, or a1 is 1 and a2 is 0.

The group -L²-Z may be a group of the following structure:

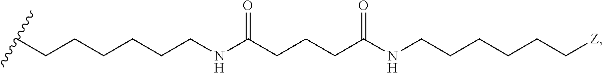

where Z is, e.g., a therapeutically active agent.

The therapeutically active agent may be a therapeutically active oligonucleotide (e.g., an antisense oligonucleotide, splice-switching oligonucleotide, siRNA, miRNA, or CpG ODN). The therapeutically active oligonucleotide may include one or more modifications. For example, the oligonucleotide may include at least one 2′-modification (e.g., 2′-methoxyethoxy) and/or at least one phosphorothioate phosphodiester. In some embodiments, in an oligonucleotide of the invention, all nucleosides are 2′-methoxyethoxy-modified nucleosides, and all internucleoside linkages are phosphorothioate phosphodiesters.

The group -L²-Z may be a group of the following structure:

[-Q³-Q⁴-Q⁵]ₛ-Z

where
s is 1 to 20;
each Q³ is independently absent, —CO—, —NH—, —O—, —S—, —SO₂—, —OC(O)—, —C(O)O—, —NHC(O)—, —C(O)NH—, —CH₂—, —CH₂NH—, —NHCH₂—, —CH₂O—, or —OCH₂—;
each Q⁴ is independently absent, optionally substituted C₁₋₁₂ alkylene, optionally substituted C₂₋₁₂ alkenylene, optionally substituted C₂₋₁₂ alkynylene, optionally substituted C₂₋₁₂ heteroalkylene, optionally substituted C₆₋₁₀ arylene, optionally substituted C₁₋₉ heteroarylene, or optionally substituted C₁₋₉ heterocyclylene;
each Q⁵ is independently absent, —CO—, —NH—, —O—, —S—, —SO₂—, —CH₂—, —C(O)O—, —OC(O)—, —C(O)NH—, —NHC(O)—, —NH—CH(R^{a})—C(O)—, —C(O)—CH(R^{a})—NH—, —OP(O)(OH)O—, or —OP(S)(OH)O—, where each R^{a} is independently H or optionally substituted C₁₋₁₂ alkyl; and
provided that at least one Q⁴ is present.

In particular preferred embodiments, each Q⁵ is independently absent, —CO—, —NH—, —O—, —S—, —SO₂—, —CH₂—, —C(O)O—, —OC(O)—, —C(O)NH—, —NHC(O)—, —OP(O)(OH)O—, or —OP(S)(OH)O—.

The group -L²-Z may be a group of the following structure:

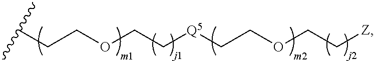

where
each of m1 and m2 is independently 0, 1, 2, 3, 4, 5, 6, 7, 8, 9, or 10; and
each of j1 and j2 is independently 1, 2, 3, 4, or 5.

The group -L²-Z may be a group of the following structure:

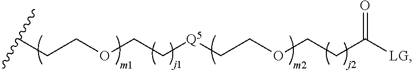

where
LG is a leaving group.

The leaving group may be pentafluorophenoxy or tetrafluorophenoxy.

The group -L²-Z may be a group of the following structure:

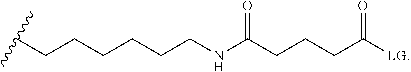

Each -L¹-T may be independently a group of the following structure:

[-Q³-Q⁴-Q⁵]ₛ-Q⁶-T,

where
s is 0 to 20;
each Q³ and each Q⁶ are independently absent, —CO—, —NH—, —O—, —S—, —SO₂—, —OC(O)—, —C(O)O—, —NHC(O)—, —C(O)NH—, —CH₂—, —CH₂NH—, —NHCH₂—, —CH₂O—, or —OCH₂—;
each Q⁴ is independently absent, optionally substituted C₁₋₁₂ alkylene, optionally substituted C₂₋₁₂ alkenylene, optionally substituted C₂₋₁₂ alkynylene, optionally substituted C₂₋₁₂ heteroalkylene, optionally substituted C₆₋₁₀ arylene, optionally substituted C₁₋₉ heteroarylene, or optionally substituted C₁₋₉ heterocyclylene; and
each Q⁵ is independently absent, —CO—, —NH—, —O—, —S—, —SO₂—, —CH₂—, —C(O)O—, —OC(O)—, —C(O)NH—, —NHC(O)—, —NH—CH(R^{a})—C(O)—, —C(O)—CH(R^{a})—NH—, —OP(O)(OH)O—, or —OP(S)(OH)O—, where each R^{a} is independently H or optionally substituted C₁₋₁₂ alkyl;
provided that at least one of Q³, Q⁴, Q⁵, and Q⁶ is present.

In particular preferred embodiments, each Q⁵ is independently absent, —CO—, —NH—, —O—, —S—, —SO₂—, —CH₂—, —C(O)O—, —OC(O)—, —C(O)NH—, —NHC(O)—, —OP(O)(OH)O—, or —OP(S)(OH)O—.

In some embodiments, s is 1, 2, 3, 4, 5, 6, 7, 8, 9, or 10.

Each -L¹-T may be independently a group of the following structure:

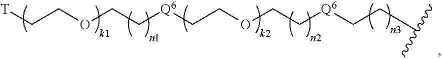

where
each of k1 and k2 is independently 0, 1, 2, 3, 4, 5, 6, 7, 8, 9, or 10; and
each of n1, n2, and n3 is independently 1, 2, 3, 4, or 5.

Each -L¹-T may be independently a group of the following structure:

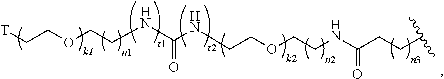

where t1 is 0 and t2 is 1, or t1 is 1 and t2 is 0.

Each -L¹-T may be a group of the following structure:

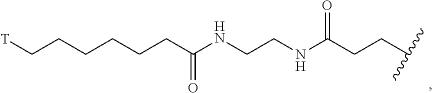

Each -L¹-T may be a group of the following structure:

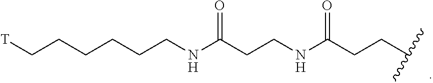

Each T may be independently a ligand (e.g., N-acetylgalactosamine). Alternatively, each T may be independently a protected ligand (e.g., N-acetylgalactosamine triacetate).

In some embodiments, Yₚ—X— is a group of the following structure:

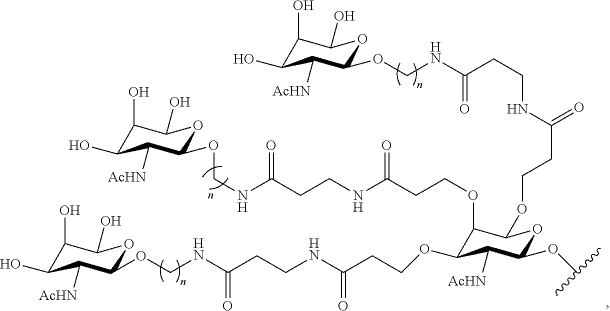

where n is 1 to 20 (e.g., 6).

In some embodiments, Yₚ—X— is a group of the following structure:

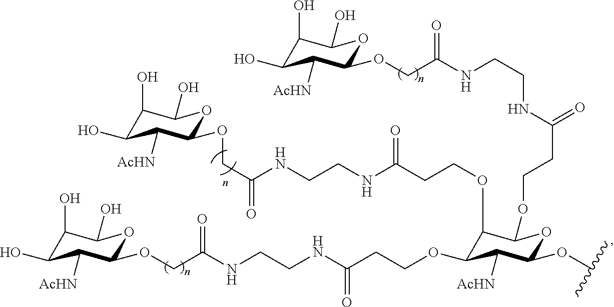

where n is 1 to 20 (e.g., 6).

The compound of the invention may be:

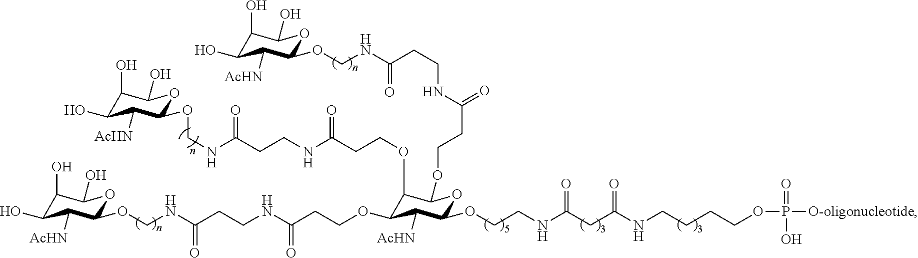

or a salt thereof,
where n is 1 to 20.

The compound of the invention may be:

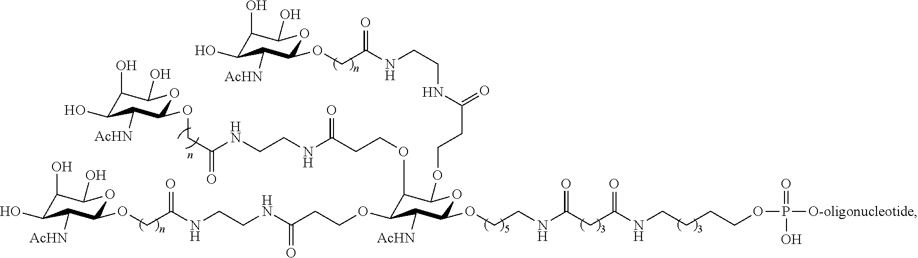

or a salt thereof,
where n is 1 to 20.

The compound of the invention may be:

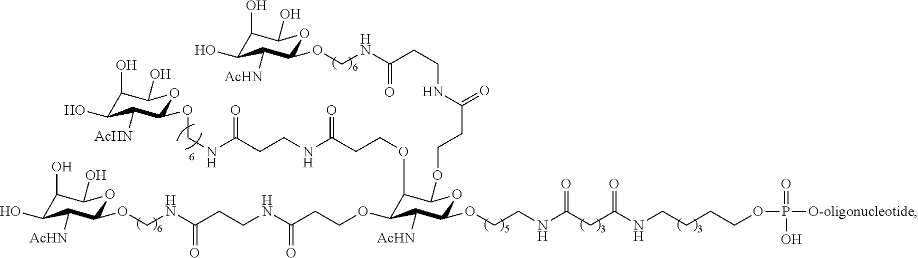

or a salt thereof.

The compound of the invention may be:

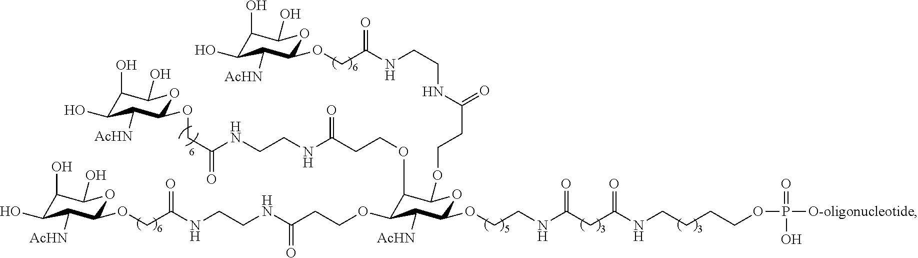

or a salt thereof.

### Hydrophobic Moieties

Advantageously, an oligonucleotide including a hydrophobic moiety may exhibit superior cellular uptake, as compared to an oligonucleotide lacking the hydrophobic moiety. Oligonucleotides including a hydrophobic moiety may therefore be used in compositions that are substantially free of transfecting agents. A hydrophobic moiety is a monovalent group (e.g., a bile acid (e.g., cholic acid, taurocholic acid, deoxycholic acid, oleyl lithocholic acid, or oleoyl cholenic acid), glycolipid, phospholipid, sphingolipid, isoprenoid, vitamin, saturated fatty acid, unsaturated fatty acid, fatty acid ester, triglyceride, pyrene, porphyrine, texaphyrine, adamantine, acridine, biotin, coumarin, fluorescein, rhodamine, Texas-Red, digoxygenin, dimethoxytrityl, t-butydimethylsilyl, t-butyldiphenylsilyl, cyanine dye (e.g., Cy3 or Cy5), Hoechst 33258 dye, psoralen, or ibuprofen) covalently linked to the oligonucleotide backbone (e.g., 5′-terminus). Non-limiting examples of the monovalent group include ergosterol, stigmasterol, β-sitosterol, campesterol, fucosterol, saringosterol, avenasterol, coprostanol, cholesterol, vitamin A, vitamin D, vitamin E, cardiolipin, and carotenoids. The linker connecting the monovalent group to the oligonucleotide may be an optionally substituted C₁₋₆₀ hydrocarbon (e.g., optionally substituted C₁₋₆₀ alkylene) or an optionally substituted C₂₋₆₀ heteroorganic (e.g., optionally substituted C₂₋₆₀ heteroalkylene), where the linker may be optionally interrupted with one, two, or three instances independently selected from the group consisting of an optionally substituted arylene, optionally substituted heterocyclylene, and optionally substituted cycloalkylene. The linker may be bonded to an oligonucleotide through, e.g., an oxygen atom attached to a 5′-terminal carbon atom, a 3′-terminal carbon atom, a 5′-terminal phosphate or phosphorothioate, a 3′-terminal phosphate or phosphorothioate, or an internucleoside linkage.

### Cell Penetrating Peptides

One or more cell penetrating peptides (e.g., from 1 to 6 or from 1 to 3) can be attached to an oligonucleotide disclosed herein as an auxiliary moiety. The CPP can be linked to the oligonucleotide through a disulfide linkage, as disclosed herein. Thus, upon delivery to a cell, the CPP can be cleaved intracellularly, e.g., by an intracellular enzyme (e.g., protein disulfide isomerase, thioredoxin, or a thioesterase) and thereby release the polynucleotide.

CPPs are known in the art (e.g., TAT or Arg₈) (Snyder and Dowdy, 2005, Expert Opin. Drug Deliv. 2, 43-51). Specific examples of CPPs including moieties suitable for conjugation to the oligonucleotides disclosed herein are provided, e.g., in WO 2015/188197; the disclosure of these CPPs is incorporated by reference herein.

CPPs are positively charged peptides that are capable of facilitating the delivery of biological cargo to a cell. It is believed that the cationic charge of the CPPs is essential for their function. Moreover, the transduction of these proteins does not appear to be affected by cell type, and these proteins can efficiently transduce nearly all cells in culture with no apparent toxicity. In addition to full-length proteins, CPPs have also been used successfully to induce the intracellular uptake of DNA, antisense polynucleotides, small molecules, and even inorganic 40 nm iron particles suggesting that there is considerable flexibility in particle size in this process.

A CPP useful in the methods and compositions of the invention may include a peptide featuring substantial alpha-helicity. It has been discovered that transfection is optimized when the CPP exhibits significant alpha-helicity. In another embodiment, the CPP includes a sequence containing basic amino acid residues that are substantially aligned along at least one face of the peptide. A CPP useful in the invention may be a naturally occurring peptide or a synthetic peptide.

### Polymers

An oligonucleotide of the invention may include covalently attached neutral polymer-based auxiliary moieties. Neutral polymers include poly(C₁₋₆alkylene oxide), e.g., poly(ethylene glycol) and poly(propylene glycol) and copolymers thereof, e.g., di- and triblock copolymers. Other examples of polymers include esterified poly(acrylic acid), esterified poly(glutamic acid), esterified poly(aspartic acid), poly(vinyl alcohol), poly(ethylene-co-vinyl alcohol), poly(N-vinyl pyrrolidone), poly(ethyloxazoline), poly(alkylacrylates), poly(acrylamide), poly(N-alkylacrylamides), poly(N-acryloylmorpholine), poly(lactic acid), poly(glycolic acid), poly(dioxanone), poly(caprolactone), styrene-maleic acid anhydride copolymer, poly(L-lactide-co-glycolide) copolymer, divinyl ether-maleic anhydride copolymer, N-(2-hydroxypropyl)methacrylamide copolymer (HMPA), polyurethane, N-isopropylacrylamide polymers, and poly(N,N-dialkylacrylamides). Exemplary polymer auxiliary moieties may have molecular weights of less than 100, 300, 500, 1000, or 5000 Da (e.g., greater than 100 Da). Other polymers are known in the art.

### Internucleoside Linkage Modifications

Oligonucleotides of the invention may include one or more internucleoside linkage modifications. The two main classes of internucleoside linkages are defined by the presence or absence of a phosphorus atom. Non-limiting examples of phosphorus-containing internucleoside linkages include phosphodiester linkages, phosphotriester linkages, phosphorothioate diester linkages, phosphorothioate triester linkages, morpholino internucleoside linkages, methylphosphonates, and phosphoramidate. Non-limiting examples of non-phosphorus internucleoside linkages include methylenemethylimino (—CH₂—N(CH₃)—O—CH₂—), thiodiester (—O—C(O)—S—), thionocarbamate (—O—C(O)(NH)—S—), siloxane (—O—Si(H)₂—O—), and N,N′-dimethylhydrazine (—CH2—N(CH₃)—N(CH₃)—). Modified linkages, compared to natural phosphodiester linkages, can be used to alter, typically increase, nuclease resistance of the oligonucleotide. Methods of preparation of phosphorous-containing and non-phosphorous-containing internucleoside linkages are known in the art.

Internucleoside linkages may be stereochemically enriched. For example, phosphorothioate-based internucleoside linkages (e.g., phosphorothioate diester or phosphorothioate triester) may be stereochemically enriched. The stereochemically enriched internucleoside linkages including a stereogenic phosphorus are typically designated S_{P} or R_{P} to identify the absolute stereochemistry of the phosphorus atom. Within an oligonucleotide, S_{P} phosphorothioate indicates the following structure:

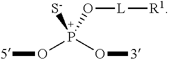

Within an oligonucleotide, R_{P} phosphorothioate indicates the following structure:

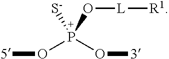

The oligonucleotides of the invention may include one or more neutral internucleoside linkages. Non-limiting examples of neutral internucleoside linkages include phosphotriesters, phosphorothioate triesters, methylphosphonates, methylenemethylimino (5′-CH₂—N(CH₃)—O-3′), amide-3 (5′-CH₂—C(═O)—N(H)-3′), amide-4 (5′-CH₂—N(H)—C(═O)-3′), formacetal (5′-O—CH₂—O-3′), and thioformacetal (5′-S—CH₂—O-3′). Further neutral internucleoside linkages include nonionic linkages including siloxane (dialkylsiloxane), carboxylate ester, carboxamide, sulfide, sulfonate ester, and amides (See for example: Carbohydrate Modifications in Antisense Research; Y. S. Sanghvi and P. D. Cook, Eds., ACS Symposium Series 580; Chapters 3 and 4, 40-65).

An internucleoside linkage modification may include a targeting moiety as described herein.

### Terminal Modifications

Oligonucleotides of the invention may include a terminal modification, e.g., a 5′-terminal modification or a 3′-terminal modification.

The 5′ end of an oligonucleotide may be, e.g., hydroxyl, a hydrophobic moiety, a targeting moiety, 5′ cap, phosphate, diphosphate, triphosphate, phosphorothioate, diphosphorothioate, triphosphorothioate, phosphorodithioate, diphosphrodithioate, triphosphorodithioate, phosphonate, phosphoramidate, a cell penetrating peptide, an endosomal escape moiety, or a neutral organic polymer. An unmodified 5′-terminus is hydroxyl or phosphate. An oligonucleotide having a 5′ terminus other than 5′-hydroxyl or 5′-phosphate has a modified 5′ terminus.

The 3′ end of an oligonucleotide may be, e.g., hydroxyl, a targeting moiety, a hydrophobic moiety, phosphate, diphosphate, triphosphate, phosphorothioate, diphosphorothioate, triphosphorothioate, phosphorodithioate, disphorodithioate, triphosphorodithioate, phosphonate, phosphoramidate, a cell penetrating peptide, an endosomal escape moiety, or a neutral organic polymer (e.g., polyethylene glycol). An unmodified 3′-terminus is hydroxyl or phosphate. An oligonucleotide having a 3′ terminus other than 3′-hydroxyl or 3′-phosphate has a modified 3′ terminus.

The terminal modification (e.g., 5′-terminal modification) may include a targeting moiety as described herein.

The terminal modification (e.g., 5′-terminal modification) may include a hydrophobic moiety as described herein.

### Methods of the Invention

Compounds of the invention may be used to deliver a therapeutically active agent to a cell having one or more surface receptors using methods of the invention. The method of the invention may include contacting the cell with the compound of the invention, or a salt thereof, where at least one T is a ligand targeting the one or more surface receptors, and Z is a therapeutically active agent. The cell may be in a tissue. The tissue may be in a subject.

Compounds of the invention may be prepared by reacting a compound of the invention having a conjugation moiety (e.g., Z is a conjugation moiety) with a compound of formula (III):

Z¹—Z², (III)

or a salt thereof,
where
Z¹ is a complementary conjugation moiety (e.g., complementary to Z); and
Z² is a therapeutically active agent.

The resulting product (e.g., one in which each T is a protected ligand) may be deprotected to produce a compound of the invention in which Z is a therapeutically active agent and at least one T is a ligand.

The following examples are meant to illustrate the invention. They are not meant to limit the invention in any way.

### EXAMPLES

### Example 1. Preparation of a Targeting Moiety of the Invention

In general, a targeting moiety of the invention may be prepared using techniques and methods known in the art and those described herein. For example, a targeting moiety may be prepared according to the procedure illustrated in Schemes 1, 2, and 3 and described herein.

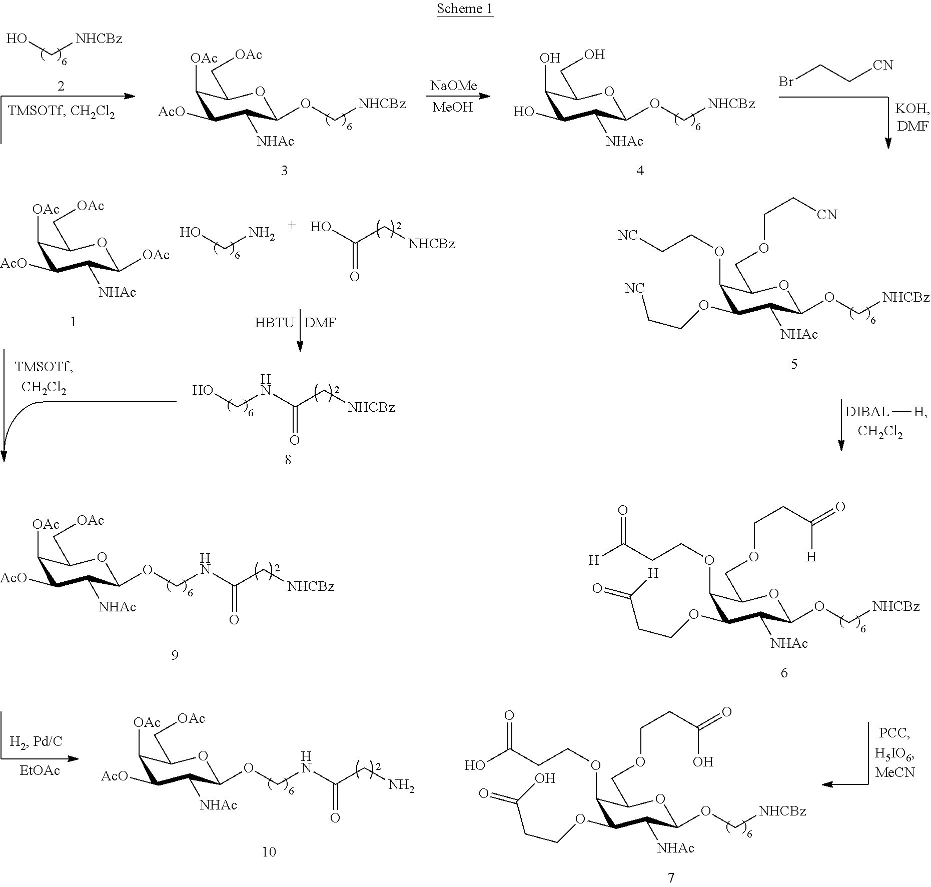

Preparation of compound 3: compound 1 was dissolved in DCM with compound 2 (0.9 equiv.). TMSOTf (1.0 equiv.) was added dropwise at room temperature, and the resulting mixture was stirred for 16 hours. Then, the reaction mixture was washed with 5% aqueous NaHCO₃, stirred for 30 minutes, and separated, and the organic phase was collected. The organic phase was then extracted with dichloromethane (DCM) and concentrated to dryness. The product was recrystallized from 2:1 EtOAc/hexane to yield a white solid (83% yield).

Preparation of compound 4: compound 3 was dissolved in 1:1 methanol:CH₂Cl₂. NaOMe (0.11 equiv.) was added, and the resulting mixture was stirred under nitrogen for one hour at room temperature. The reaction mixture was concentration in vacuo to produce a while solid, which was used in the next step without further purification.

Preparation of compound 5: under inert atmosphere, 3-bromopropionitrile (12.1 equiv.) was added dropwise at 0° C. to a DMF solution of compound 4 (1.0 equiv.), and KOH (8.1 equiv.). The resulting mixture was gradually warmed to room temperature and stirred overnight. The mixture was then concentrated to give a residue, which was dissolve in EtOAc and washed with brine. The organic layer was dried over Na₂SO₄, concentrated, and subjected to silica gel chromatography (3:2 EtOAc/hexanes) to give the product.

Preparation of compound 6: to a stirred suspension of compound 5 in anhydrous CH₂Cl₂ at −70° C., DIBAL-H (1.0M) in CH₂Cl₂ may be added dropwise. The resulting mixture may then be stirred under inert atmosphere for 2 hours. The reaction mixture may be worked up using Fieser procedure to remove aluminum byproducts. First, the reaction mixture may be diluted with ether and warmed to 0° C. The imine intermediate may be hydrolyzed by slow addition of water. Then, 15% aqueous NaOH may be added, followed by water. The resulting mixture may be warmed to room temperature and stirred for 15 minutes, at which time, anhydrous MgSO₄ may be added. The resulting mixture may be stirred for 15 minutes and filtered through a Celite® pad. The product may be purified by silica gel chromatography (3:2 EtOAc/hexanes).

Preparation of compound 7: periodic acid may be added to MeCN and stirred vigorously for 15 minutes at room temperature. Compound 6 may then be added, followed by pyridinium chlorochromate (PCC) in MeCN in 2 parts. After 3 hours stirring, the reaction mixture may be diluted with EtOAc and washed with brine, NaHCO₃, brine, and dried over Na₂SO₄. The separated organic layer may be concentrated in vacuo to give compound 7. A quantitative yield is expected for this reaction.

Preparation of compound 8: CBz-protected β-alanine (1 equiv.) and HBTU (1.1 equiv.) were dissolved in DMF. The resulting solution was cooled to 0-10° C., and N,N-diisopropyl-N-ethylamine (DIPEA, 1.5 equiv. was added dropwise. The resulting mixture was stirred for at least 30 minutes at 0-10° C. and then cooled to −25° C. 6-amino-1-hexanol (1 equiv.) in DMF was added dropwise. After 4 hours, the reaction was quenched with water, and the resulting mixture was stirred for 1 hour and filtered, and the filter cake was washed with water. A slurry of the cake and water was filtered twice. The filter cake was dried under vacuum at 40° C. until water content was 0.3% or less (76% yield).

Preparation of compound 9: compound 1 (1.1 equiv.) was dissolved in DCM, and the resulting solution was cooled to 5-15° C. TMSOTf (1.2 equiv.) was added, and the resulting mixture was stirred for 2 hours at 5-15° C. Compound 8 (1.0 equiv.) was added to the reaction mixture, and the resulting mixture was stirred for 16 hours as 30-40° C. The reaction mixture was then cooled to 15-25° C. Water was added, and the mixture was stirred for 10 minutes. Layers were separated, and the organic phase was washed with water twice. The organic layer was concentrated to dryness. The product was recrystallized from 2:1 EtOAc/hexane and filtered, and the filter cake was dried in vacuo to product the white solid (65% yield).

Preparation of compound 10: compound 9 was dissolved in ethyl acetate (EtOAc) under nitrogen, and trifluoroacetic acid (1.5 equiv) and Pd/C (20% (w/w)) were added with stirring. Hydrogen gas (balloon) was added to the reaction at 2 atm, and the resulting mixture was stirred at room temperature for 2 hours. Solid Pd/C was filtered through a pad of Celite®, and the filtrate was concentrated in vacuo to give a crude product, which may be used without purification in the next step (coupling to the tri-perfluorophenyl ester of compound 7).

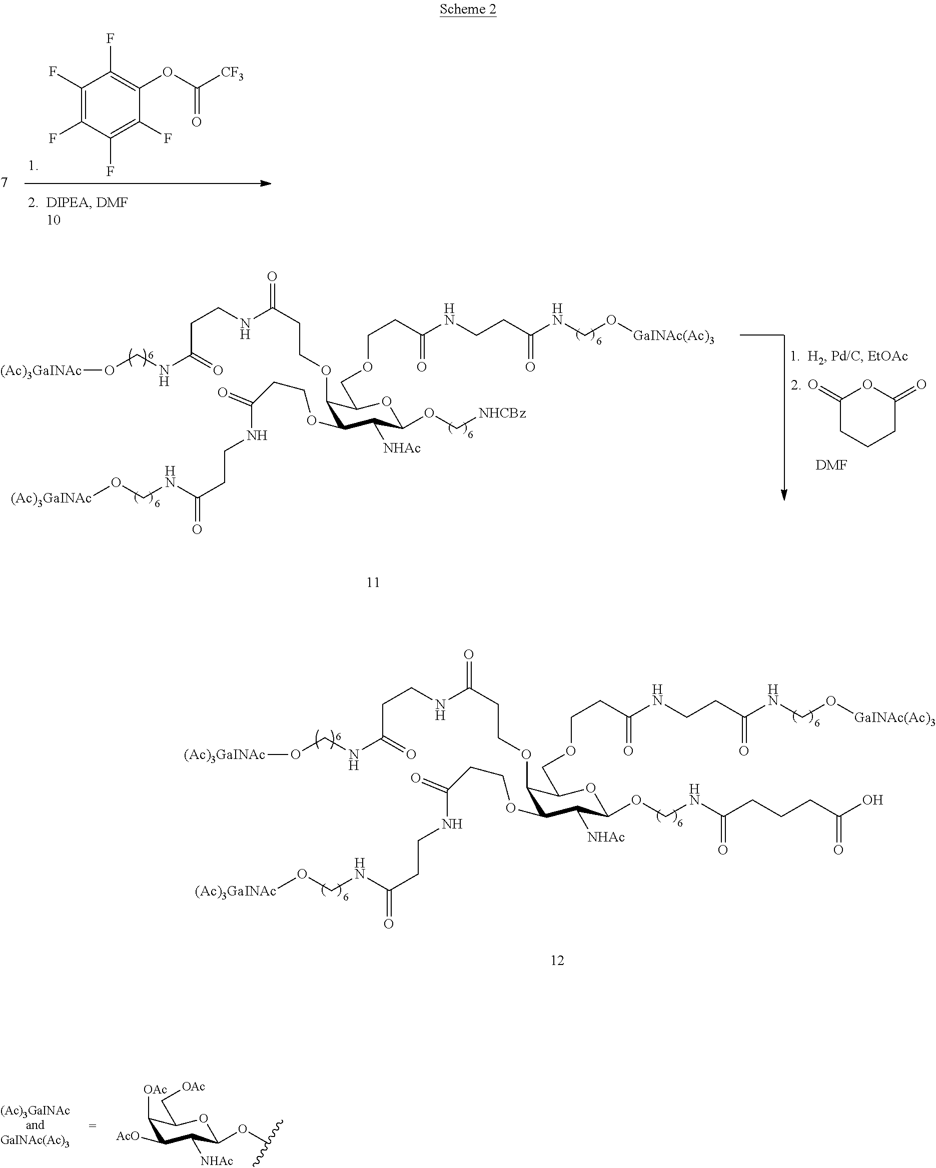

Preparation of compound 11: compound 7 (1.0 equiv.) may be dissolved in CH₂Cl₂ at 0-10° C. To this solution of compound 7, DIPEA (8 equiv.) and perfluorophenyl trifluoroacetate (4 equiv.) may be added. The resulting mixture may be stirred for 2 hours at 0-10° C. and may be washed with water at 0-10° C., and the separated organic phase may be dried over Na₂SO₄ (200% (w/w)). The organic phase may be cooled to 0-10° C., DIPEA (3 equiv.) may be added, compound 10 (3.4 equiv.) in CH₂Cl₂ may be added dropwise, and the resulting mixture may be stirred for 1 hour at 0-10° C. The reaction mixture may be washed with saturated aqueous NH₄Cl at 0-10° C., phases may be separate, and the organic phase may be washed with water, dried over Na₂SO₄ (200% (w/w)), filtered, and concentrated. To the concentrated filtrate, MTBE may be added to precipitate the solid from the remaining CH₂Cl₂/MTBE.

Removal of the CBz protecting group in 11: this reaction may be performed under the same hydrogenation conditions as those described for the preparation of compound 10, with the exception that the crude product may be dissolved in CH₂Cl₂. The resulting solution may be added dropwise to MTBE to precipitate solid product, which may be filtered. The filter cake may then be combined with 50% (w/w) Al₂O₃ in CH₂Cl₂ at 20-25° C. for 30 minutes. The resulting mixture may be filtered, and the filtrate may be dried to give the desired product as a solid.

Preparation of compound 12: the product of CBz removal from 11 may be dissolved in DMF and stirred at room temperature for 4 hours with glutaric anhydride. The reaction mixture may be washed with saturated aqueous NaHCO₃, layers may be separated, and the organic phase may be washed with CH₂Cl₂. The resulting solution may be dried in vacuo to give the product.

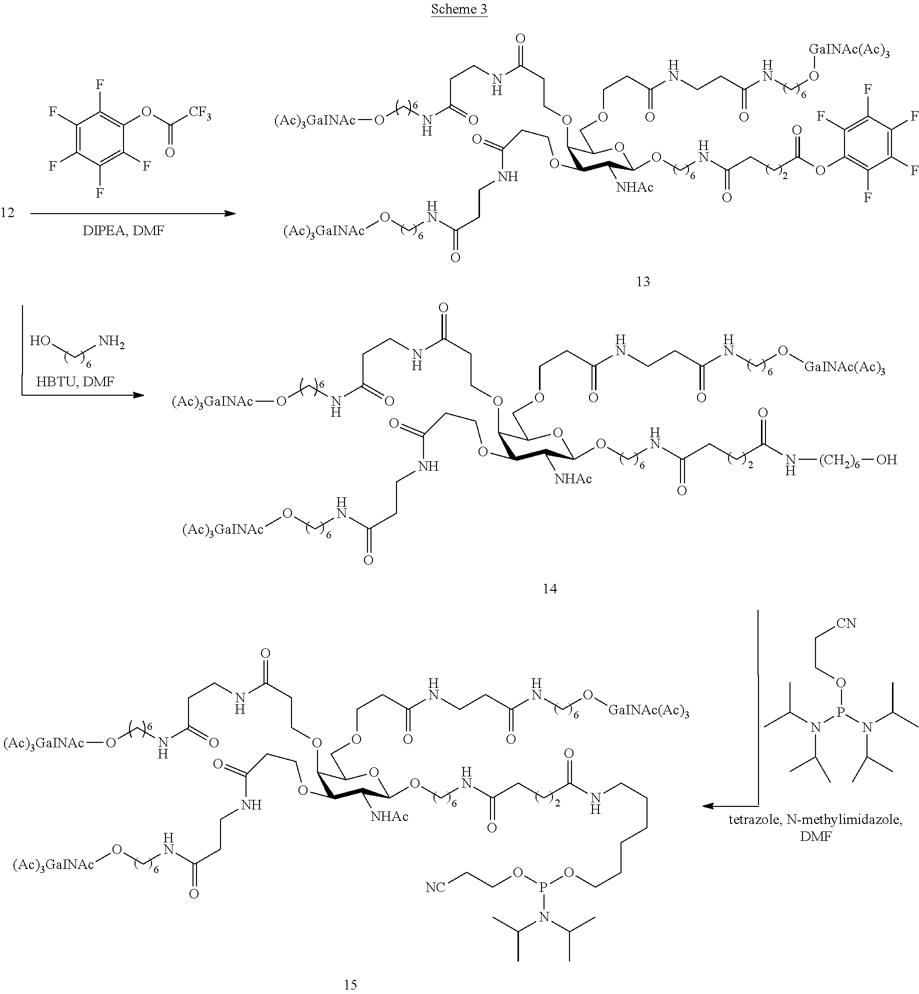

Preparation of compound 13: compound 12 (1 equiv.) may be dissolved in CH₂Cl₂ at 0-10° C. DIPEA (2.0 equiv.) and perfluorophenyl trifluoroacetate (1.5 equiv.) may be added. The reaction mixture may be stirred for 2 hours at 0-10° C. and washed with water at 0-10° C., and the separated organic phase may be dried over Na₂SO₄ (200% (w/w)) and filtered. The filtrate may be concentrated, and the product may be isolated as a solid from CH2Cl₂/MTB.

Preparation of compound 14: compound 12 (1.0 equiv.) and HBTU (1.1 equiv.) may be dissolved in CH₂Cl₂. The resulting solution may be stirred and cooled to 0-10° C. DIPEA (1.5 equiv.) may be added, and the resulting mixture may be stirred at 0-10° C. for 15 minutes, at which time, 6-amino-1-hexanol (1.05 equiv.) in CH₂Cl₂ may be added dropwise, and the reaction mixture may be stirred for 1 hour at 0-10° C. CH₂Cl₂ may be added to the reaction mixture, followed by the addition of aqueous saturated NH₄Cl at 0-10° C. Layers may be separated, and the organic phase may be washed with NH₄Cl, dried over Na₂SO₄ (200% (w/w)), filtered, and concentrated. To the concentrated filtrate, MTBE may be added to precipitate the solid from CH₂Cl₂/MTBE. The resulting mixture may be filtered, and the filter cake may be dissolved in CH₂Cl₂. To the resulting solution, Al₂O₃ (100% (w/w)) may be added, and the resulting mixture may be stirred for an hour, at which time, the mixture may be filtered, and the filtrate may be dried in vacuo to give the product as a solid.

Preparation of compound 15: Compound 14 (1.0 equiv.), N-methylimidazole (0.2 equiv.), and tetrazole (0.8 equiv.) may be dissolved in DMF. The resulting solution may be stirred and cooled to 0-10° C. 2-Cyanoethyl-N,N,N′,N′-tetraisopropylphosphordiamidite (3.0 equiv.) may be added dropwise, and the resulting mixture may be stirred for 1 hour at room temperature. The reaction may be quenched by the dropwise addition of water at 0-10° C. Saturated aqueous NaCl and EtOAc may be added at 0-10° C. Layers may be separated, and the aqueous phase may be extracted with EtOAc twice. The organic phase may be dried over Na₂SO₄, filtered, and concentrated. The product may be isolated as a solid by precipitation from CH2Cl₂/MTBE.

Compound 13 and compound 15 may be used in the preparation of compounds of the invention described herein.

### Example 2

Compound 15 from Example 1 may be coupled to an oligonucleotide to produce compound 16.

### GC

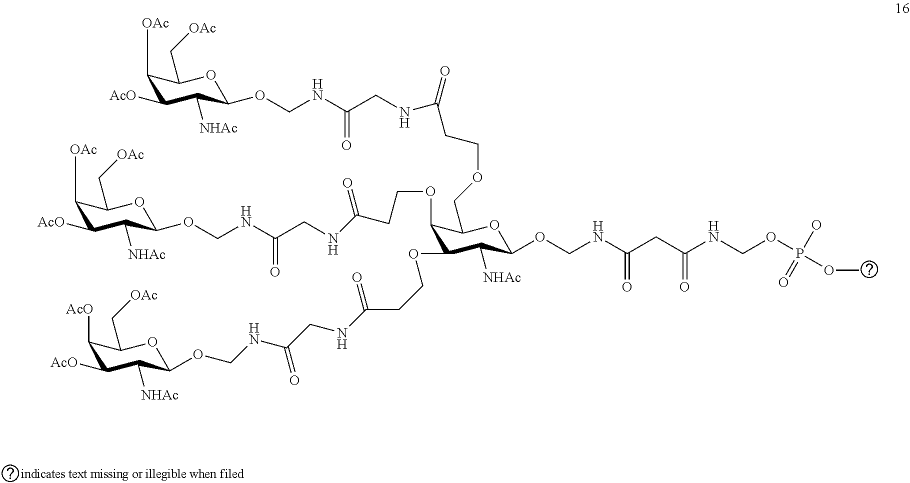

For example, reaction between compound 15 and oligo-O—P(O)(OH)—O—(CH₂)₆—NH₂, or a salt thereof, in buffered medium (e.g., sodium tetraborate buffer at pH 8.5) may produce compound 16.

### Example 3

Additionally, a targeting moiety may be prepared as shown in Scheme 4 and described below, e.g., from compound 11 in Example 1.

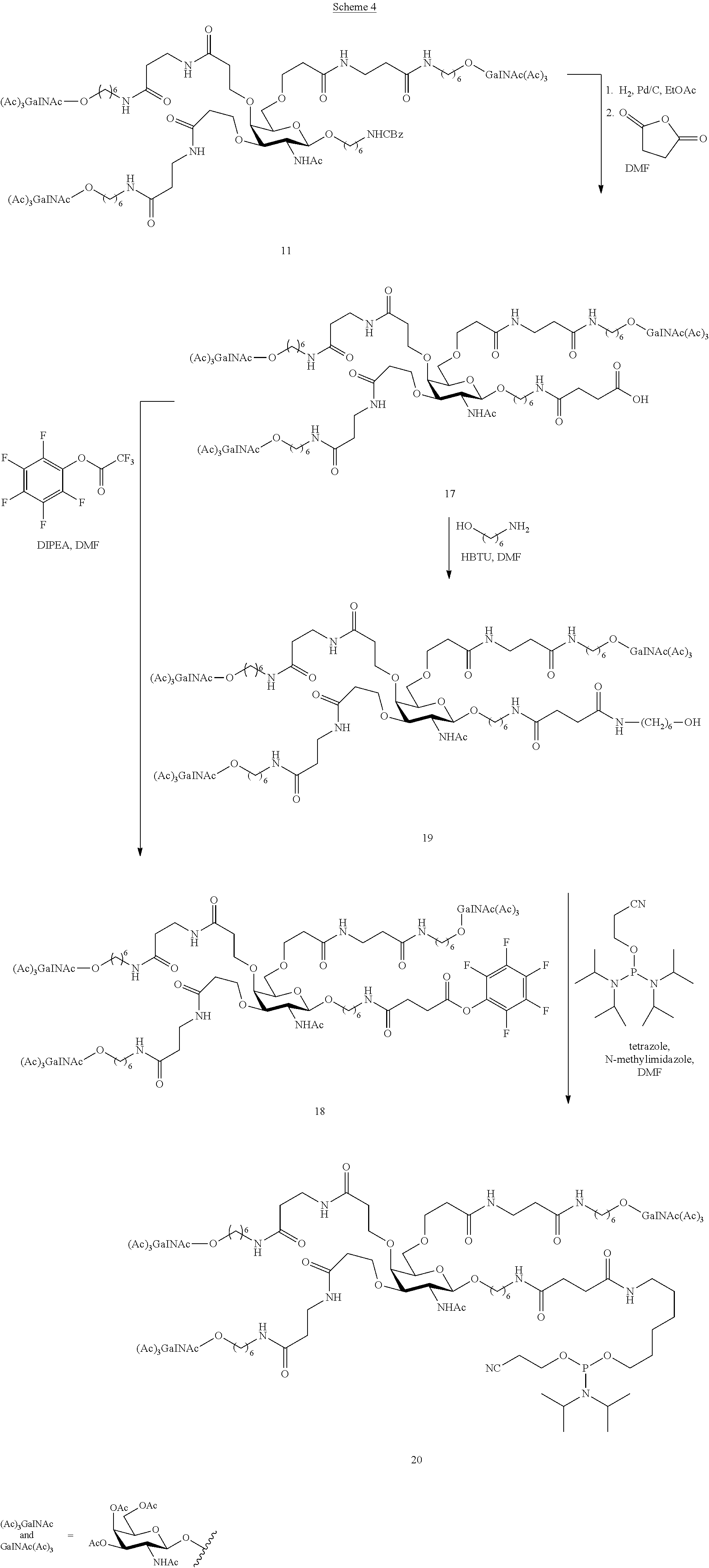

Removal of the CBz protecting group in 11: this reaction may be performed under the same hydrogenation conditions as those described for the preparation of compound 10, with the exception that the crude product may be dissolved in CH₂Cl₂. The resulting solution may be added dropwise to MTBE to precipitate solid product, which may be filtered. The filter cake may then be combined with 50% (w/w) Al₂O₃ in CH₂Cl₂ at 20-25° C. for 30 minutes. The resulting mixture may be filtered, and the filtrate may be dried to give the desired product as a solid.

Preparation of compound 17: the product of CBz removal from 11 may be dissolved in DMF and stirred at room temperature for 4 hours with succinic anhydride. The reaction mixture may be washed with saturated aqueous NaHCO₃, layers may be separated, and the organic phase may be washed with CH₂Cl₂. The resulting solution may be dried in vacuo to give the product.

Preparation of compound 18: compound 17 (1 equiv.) may be dissolved in CH₂Cl₂ at 0-10° C. DIPEA (2.0 equiv.) and perfluorophenyl trifluoroacetate (1.5 equiv.) may be added. The reaction mixture may be stirred for 2 hours at 0-10° C. and washed with water at 0-10° C., and the separated organic phase may be dried over Na₂SO₄ (200% (w/w)) and filtered. The filtrate may be concentrated, and the product may be isolated as a solid from CH₂Cl₂/MTBE.

Preparation of compound 19: compound 17 (1.0 equiv.) and HBTU (1.1 equiv.) may be dissolved in CH₂Cl₂. The resulting solution may be stirred and cooled to 0-10° C. DIPEA (1.5 equiv.) may be added, and the resulting mixture may be stirred at 0-10° C. for 15 minutes, at which time, 6-amino-1-hexanol (1.05 equiv.) in CH₂Cl₂ may be added dropwise, and the reaction mixture may be stirred for 1 hour at 0-10° C. CH₂Cl₂ may be added to the reaction mixture, followed by the addition of aqueous saturated NH₄Cl at 0-10° C. Layers may be separated, and the organic phase may be washed with NH₄Cl, dried over Na₂SO₄ (200% (w/w)), filtered, and concentrated. To the concentrated filtrate, MTBE may be added to precipitate the solid from CH₂Cl₂/MTBE. The resulting mixture may be filtered, and the filter cake may be dissolved in CH₂Cl₂. To the resulting solution, Al₂O₃ (100% (w/w)) may be added, and the resulting mixture may be stirred for an hour, at which time, the mixture may be filtered, and the filtrate may be dried in vacuo to give the product as a solid.

Preparation of compound 20: Compound 19 (1.0 equiv.), N-methylimidazole (0.2 equiv.), and tetrazole (0.8 equiv.) may be dissolved in DMF. The resulting solution may be stirred and cooled to 0-10° C. 2-Cyanoethyl-N,N,N′,N′-tetraisopropylphosphordiamidite (3.0 equiv.) may be added dropwise, and the resulting mixture may be stirred for 1 hour at room temperature. The reaction may be quenched by the dropwise addition of water at 0-10° C. Saturated aqueous NaCl and EtOAc may be added at 0-10° C. Layers may be separated, and the aqueous phase may be extracted with EtOAc twice. The organic phase may be dried over Na₂SO₄, filtered, and concentrated. The product may be isolated as a solid by precipitation from CH₂Cl₂/MTBE.

Compound 18 and compound 20 may be used in the preparation of compounds of the invention described herein.

### Other Embodiments

Various modifications and variations of the described invention will be apparent to those skilled in the art without departing from the scope and spirit of the invention. Although the invention has been described in connection with specific embodiments, it should be understood that the invention as claimed should not be unduly limited to such specific embodiments. Indeed, various modifications of the described modes for carrying out the invention that are obvious to those skilled in the art are intended to be within the scope of the invention.

Other embodiments are in the claims.

What is claimed is:

1. A compound of formula (I):
Yₚ—X-L²-Z, (I)
or a salt thereof,
wherein
p is 1 to 5;
X is a monosaccharide;
each Y is independently -L¹-T, H, protecting group, optionally substituted hydrocarbon, or optionally substituted heteroorganic group, wherein each T is independently a ligand or a protected ligand, and each L¹ is independently a covalent linker;
L² is a conjugation linker;
Z is a therapeutically active agent, protecting group, or a conjugation moiety;
provided that at least one Y is -L¹-T.

2. The compound of claim 1, or a salt thereof, wherein the monosaccharide is a pentose or hexose, wherein,
when the monosaccharide is a pentose, p is 1 to 3, and
when the monosaccharide is a hexose, p is 1 to 4.

3. The compound of claim 1 or 2, or a salt thereof, wherein the monosaccharide is N-acetylgalactosamine, galactosamine, galactose, mannose, allose, altrose, glucose, gulose, idose, talose, arabinose, lyxose, ribose, or xylose.

4. The compound of claim 3, or a salt thereof, wherein the monosaccharide is N-acetylgalactosamine.

5. The compound of any one of claims 1 to 4, or a salt thereof, wherein -L²-Z is a group of the following structure:
-Q¹-Q²-Z,
wherein
Q¹ is [-Q³-Q⁴-Q⁵]ₛ-Q^{C}-B¹, wherein B¹ is a bond to Q²;
Q² is [-Q³-Q⁴-Q⁵]ₛ-B², where B² is a bond to Z;
each Q³ is independently absent, —CO—, —NH—, —O—, —S—, —SO₂—, —OC(O)—, —C(O)O—, —NHC(O)—, —C(O)NH—, —CH₂—, —CH₂NH—, —NHCH₂—, —CH₂O—, or —OCH₂—;
each Q⁴ is independently absent, optionally substituted C₁₋₁₂ alkylene, optionally substituted C₂₋₁₂ alkenylene, optionally substituted C₂₋₁₂ alkynylene, optionally substituted C₂₋₁₂ heteroalkylene, optionally substituted C₆₋₁₀ arylene, optionally substituted C₁₋₉ heteroarylene, or optionally substituted C₁₋₉ heterocyclylene;
each Q⁵ is independently absent, —CO—, —NH—, —O—, —S—, —SO₂—, —CH₂—, —C(O)O—, —OC(O)—, —C(O)NH—, —NHC(O)—, —NH—CH(R^{a})—C(O)—, —C(O)—CH(R^{a})—NH—, —OP(O)(OH)O—, or —OP(S)(OH)O—, wherein each R^{a} is independently H or optionally substituted C₁₋₁₂ alkyl;
Q^{C} is optionally substituted C₂₋₁₂ alkylene, optionally substituted C₂₋₁₂ heteroalkylene, optionally substituted C₁₋₁₂ thioheterocyclylene, optionally substituted C₁₋₁₂ heterocyclylene, cyclobut-3-ene-1,2-dione-3,4-diyl, pyrid-2-yl hydrazone, optionally substituted C₆₋₁₆ triazoloheterocyclylene, optionally substituted C₈₋₁₆ triazolocycloalkenylene, or a dihydropyridazine group; and
each s is independently 0 to 20.

6. The compound of claim 5, or a salt thereof, wherein -L²-Z is a group of the following structure: 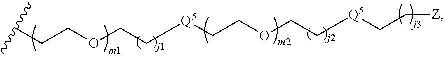
wherein
each of m1 and m2 is independently 0, 1, 2, 3, 4, 5, 6, 7, 8, 9, or 10; and
each of j1, j2, and j3 is independently 1, 2, 3, 4, or 5.

7. The compound of claim 5 or 6, or a salt thereof, wherein each Q⁵ is independently absent, —CO—, —NH—, —O—, —S—, —SO₂—, —CH₂—, —C(O)O—, —OC(O)—, —C(O)NH—, —NHC(O)—, —OP(O)(OH)O—, or —OP(S)(OH)O—

8. The compound of claim 5 or 6, or a salt thereof, wherein each Q⁵ is independently NHC(O)— or —C(O)NH—.

9. The compound of claim 8, or a salt thereof, wherein -L²-Z is a group of the following structure: 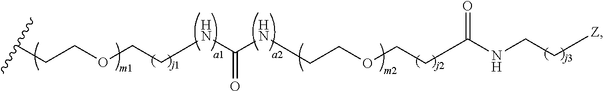
wherein a1 is 0 and a2 is 1, or a1 is 1 and a2 is 0.

10. The compound of claim 9, or a salt thereof, wherein -L²-Z is a group of the following structure:

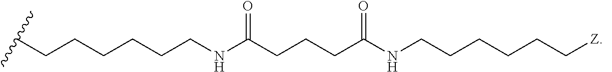

11. The compound of claim 9, or a salt thereof, wherein -L²-Z is a group of the following structure:

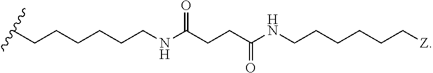

12. The compound of any one of claims 1 to 11, or a salt thereof, wherein Z is a therapeutically active agent.

13. The compound of claim 12, or a salt thereof, wherein the therapeutically active agent is a therapeutically active oligonucleotide.

14. The compound of claim 13, or a salt thereof, wherein the therapeutically active oligonucleotide is an antisense oligonucleotide, splice-switching oligonucleotide, siRNA, miRNA, or CpG ODN.

15. The compound of any one of claims 1 to 4, or a salt thereof, wherein -L²-Z is a group of the following structure:
[-Q³-Q⁴-Q⁵]ₛ-Z
wherein
s is 1 to 20;
each Q³ is independently absent, —CO—, —NH—, —O—, —S—, —SO₂—, —OC(O)—, —C(O)O—, —NHC(O)—, —C(O)NH—, —CH₂—, —CH₂NH—, —NHCH₂—, —CH₂O—, or —OCH₂—;
each Q⁴ is independently absent, optionally substituted C₁₋₁₂ alkylene, optionally substituted C₂₋₁₂ alkenylene, optionally substituted C₂₋₁₂ alkynylene, optionally substituted C₂₋₁₂ heteroalkylene, optionally substituted C₆₋₁₀ arylene, optionally substituted C₁₋₉ heteroarylene, or optionally substituted C₁₋₉ heterocyclylene;
each Q⁵ is independently absent, —CO—, —NH—, —O—, —S—, —SO₂—, —CH₂—, —C(O)O—, —OC(O)—, —C(O)NH—, —NHC(O)—, —NH—CH(R^{a})—C(O)—, —C(O)—CH(R^{a})—NH—, —OP(O)(OH)O—, or —OP(S)(OH)O—, wherein each R^{a} is independently H or optionally substituted C₁₋₁₂ alkyl; and
provided that at least one Q⁴ is present.

16. The compound of claim 15, or a salt thereof, wherein -L²-Z is a group of the following structure: 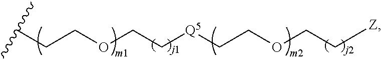
wherein
each of m1 and m2 is independently 0, 1, 2, 3, 4, 5, 6, 7, 8, 9, or 10; and
each of j1 and j2 is independently 1, 2, 3, 4, or 5.

17. The compound of claim 16, or a salt thereof, wherein -L²-Z is a group of the following structure: 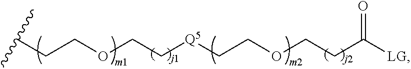
wherein
LG is a leaving group.

18. The compound of claim 17, or a salt thereof, wherein the leaving group is pentafluorophenoxy or tetrafluorophenoxy.

19. The compound of claim 17 or 18, or a salt thereof, wherein -L²-Z is a group of the following structure:

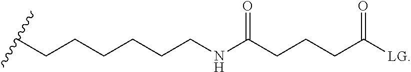

20. The compound of claim 17 or 18, or a salt thereof, wherein -L²-Z is a group of the following structure:

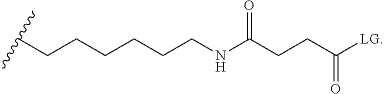

21. The compound of any one of claims 1 to 20, or a salt thereof, wherein each -L¹-T is independently a group of the following structure:
[-Q³-Q⁴-Q⁵]ₛ-Q⁶-T,
wherein
s is 0 to 20;
each Q³ and each Q⁶ are independently absent, —CO—, —NH—, —O—, —S—, —SO₂—, —OC(O)—, —C(O)O—, —NHC(O)—, —C(O)NH—, —CH₂—, —CH₂NH—, —NHCH₂—, —CH₂O—, or —OCH₂—;
each Q⁴ is independently absent, optionally substituted C₁₋₁₂ alkylene, optionally substituted C₂₋₁₂ alkenylene, optionally substituted C₂₋₁₂ alkynylene, optionally substituted C₂₋₁₂ heteroalkylene, optionally substituted C₆₋₁₀ arylene, optionally substituted C₁₋₉ heteroarylene, or optionally substituted C₁₋₉ heterocyclylene; and
each Q⁵ is independently absent, —CO—, —NH—, —O—, —S—, —SO₂—, —CH₂—, —C(O)O—, —OC(O)—, —C(O)NH—, —NHC(O)—, —NH—CH(R^{a})—C(O)—, —C(O)—CH(R^{a})—NH—, —OP(O)(OH)O—, or —OP(S)(OH)O—, wherein each R^{a} is independently H or optionally substituted C₁₋₁₂ alkyl;
provided that at least one of Q³, Q⁴, Q⁵, and Q⁶ is present.

22. The compound of claim 21, or a salt thereof, wherein s is 1, 2, 3, 4, 5, 6, 7, 8, 9, or 10.

23. The compound of claim 21 or 22, or a salt thereof, wherein each -L¹-T is independently a group of the following structure: 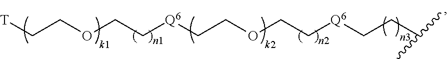
wherein
each of k1 and k2 is independently 0, 1, 2, 3, 4, 5, 6, 7, 8, 9, or 10; and
each of n1, n2, and n3 is independently 1, 2, 3, 4, or 5.

24. The compound of claim 23, or a salt thereof, wherein each Q⁶ is independently —NHC(O)— or —C(O)NH—.

25. The compound of claim 24, or a salt thereof, wherein each -L¹-T is independently a group of the following structure: 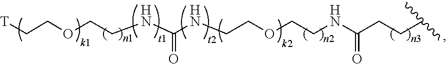
wherein t1 is 0 and t2 is 1, or t1 is 1 and t2 is 0.

26. The compound of claim 25, or a salt thereof, wherein each -L¹-T is a group of the following structure:

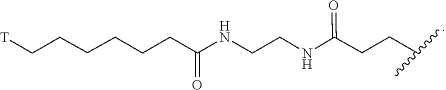

27. The compound of claim 25, or a salt thereof, wherein each -L¹-T is a group of the following structure:

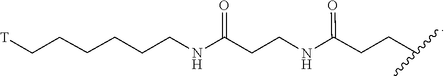

28. The compound of any one of claims 1 to 27, or a salt thereof, wherein each T is independently a ligand.

29. The compound of claim 28, or a salt thereof, wherein each T is N-acetylgalactosamine.

30. The compound of any one of claims 1 to 28, or a salt thereof, wherein each T is independently a protected ligand.

31. The compound of claim 28, or a salt thereof, wherein each T is N-acetylgalactosamine triacetate.

32. A compound of the following structure: 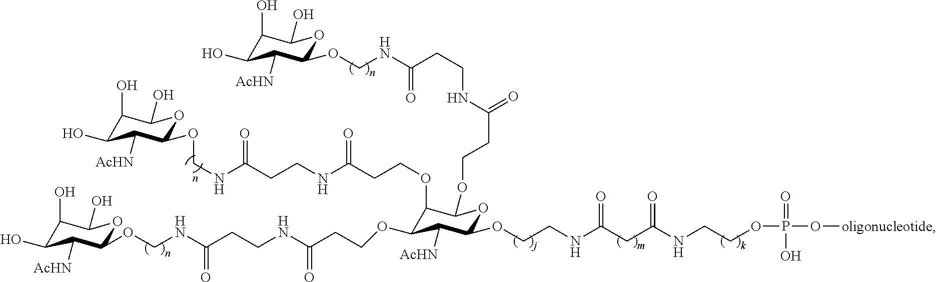
or a salt thereof,
wherein each n is independently 1 to 20, j is 1 to 11, k is 1 to 11, and m is 1 to 10.

33. The compound of claim 32, or a salt thereof, wherein j is 5.

34. The compound of claim 32 or 33, or a salt thereof, wherein k is 5.

35. The compound of any one of claims 32 to 34, or a salt thereof, wherein m is 2 or 3.

36. A compound of the following structure: 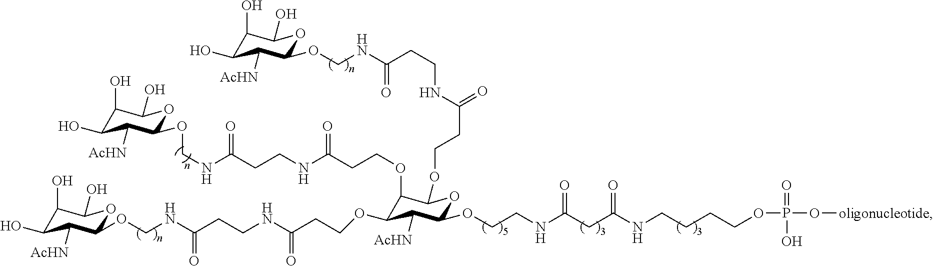
or a salt thereof,
wherein each n is independently 1 to 20.

37. The compound of claim 36, wherein the compound is: 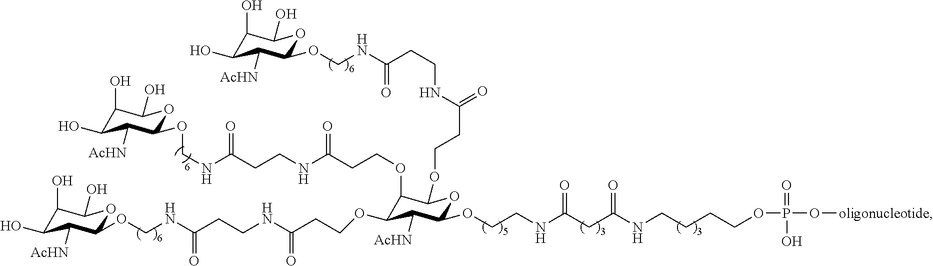
or a salt thereof.

38. A compound of the following structure: 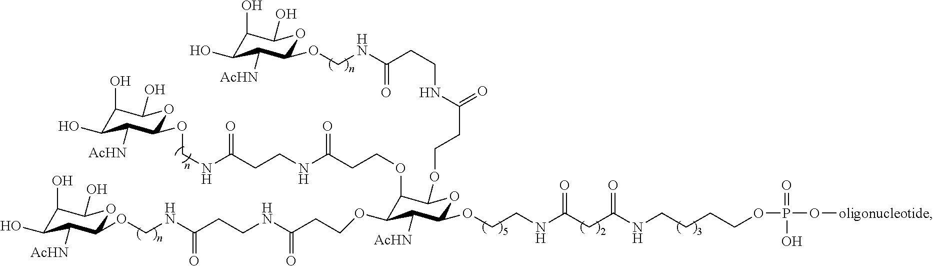
or a salt thereof,
wherein each n is independently 1 to 20.

39. The compound of claim 38, wherein the compound is: 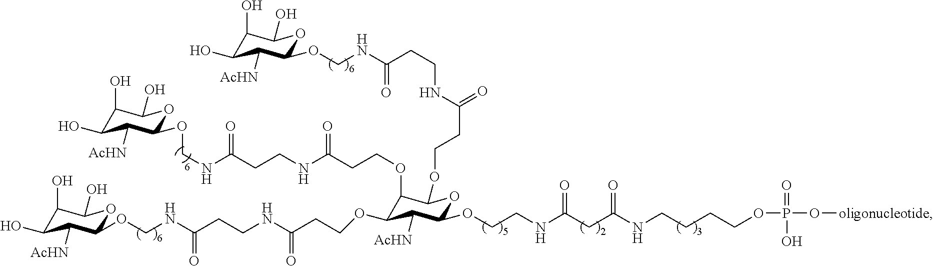
or a salt thereof.

40. A compound of the following structure: 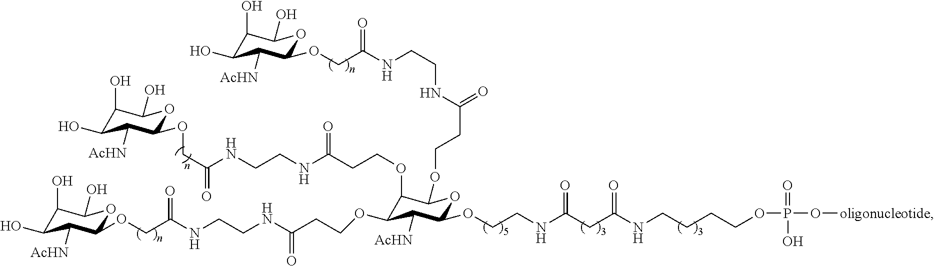
or a salt thereof,
wherein each n is independently 1 to 20.

41. The compound of claim 40, wherein the compound is: 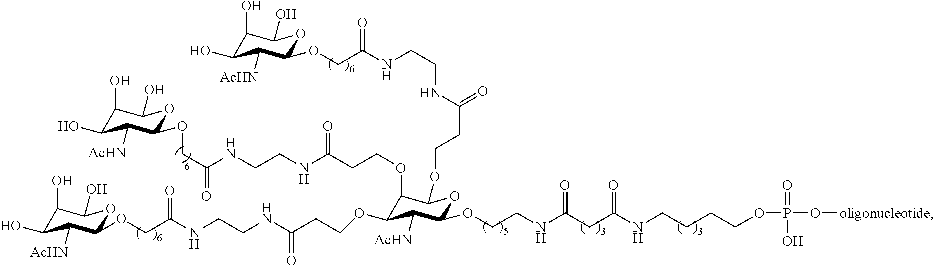
or a salt thereof.

42. A compound of the following structure: 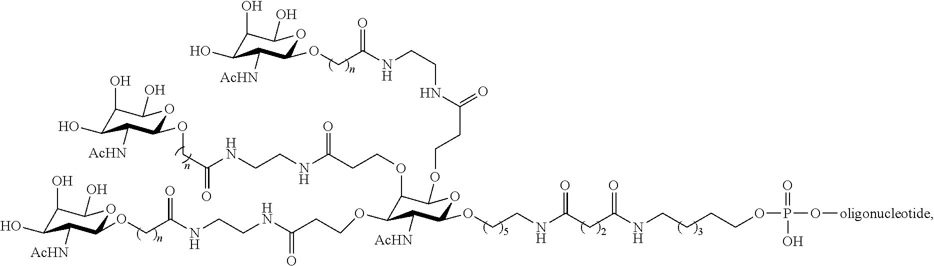
or a salt thereof,
wherein each n is independently 1 to 20.

43. The compound of claim 42, wherein the compound is: 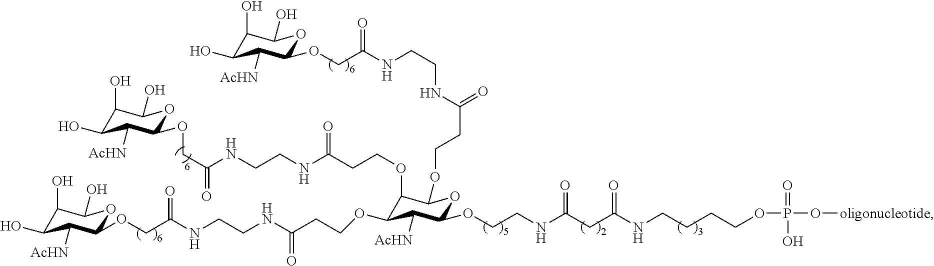
or a salt thereof.

44. A method of delivering a therapeutically active agent to a cell having one or more surface receptors, the method comprising contacting the cell with the compound of any one of claims 1 to 16 and 21 to 43, or a salt thereof, wherein at least one T is a ligand, and Z is a therapeutically active agent.

45. The method of claim 44, wherein the cell is in a tissue.

46. The method of claim 45, wherein the tissue is in a subject.

47. A method of producing the compound of claim 1, in which Z is a therapeutically active agent, the method comprising producing a product of a reaction between the compound of claim 1, in which Z is a conjugation moiety and at least one T is a protected ligand, with a compound of formula (Ill):
Z¹—Z², (III)
or a salt thereof,
wherein
Z¹ is a complementary conjugation moiety; and
Z² is a therapeutically active agent.

48. The method of claim 47, further comprising deprotecting the product to produce the compound of claim 1, in which Z is a therapeutically active agent and at least one T is a ligand.
